(19) United States
(12) Patent Application Publication
(10) Pub. No.: US 2013/0123206 A1
(43) Pub. Date: May 16, 2013

(54) TREATMENT OF METABOLIC SYNDROME AND INSULIN RESISTANCE WITH CITRUS FLAVANONES

(71) Applicants: Michael J. Quon, Bethesda, MD (US); Ranganath Muniyappa, Stafford, VA (US)

(72) Inventors: Michael J. Quon, Bethesda, MD (US); Ranganath Muniyappa, Stafford, VA (US)

(21) Appl. No.: 13/813,072

(22) PCT Filed: Jul. 29, 2011
(86) PCT No.: PCT/US11/45898

§ 371 Date: Jan. 29, 2013

Related U.S. Application Data
(60) Provisional application No. 61/369,229, filed on Jul. 30, 2010.

(51) Int. Cl. A61K 31/7034

(52) U.S. Cl. CPC A61K 31/7034
USPC 514/35

(57) ABSTRACT

Methods of treating metabolic syndrome and/or insulin resistance including administering a therapeutically effective amount of purified hesperidin, purified hesperetin, or a purified derivative or analog of either compound to a subject with metabolic syndrome or insulin resistance (alone or in the context of metabolic syndrome).

### CROSS REFERENCE TO RELATED APPLICATION

This application claims the benefit of U.S. Provisional Application No. 61/369,229, filed Jul. 30, 2010, which is incorporated herein by reference in its entirety.

### FIELD

This disclosure relates to methods for treating metabolic syndrome and insulin resistance, particularly using citrus flavanones, such as hesperidin or hesperetin.

### BACKGROUND

Diabetes, obesity, the metabolic syndrome, and their cardiovascular complications cluster together due, in part, to reciprocal relationships between insulin resistance and endothelial dysfunction (Kim et al., Circulation 113:1888-904, 2006; Muniyappa et al., Endocr Rev, 28:463-91, 2007). Indeed, therapeutic interventions in animal models and humans that improve endothelial dysfunction often simultaneously ameliorate insulin resistance (and vice-versa) (Kim et al., Circulation 113:1888-904, 2006; Muniyappa et al., Endocr Rev, 28:463-91, 2007; Potenza et al., Diabetes, 55:3594-603, 2006). Flavonoids (flavones, flavonols, flavanones and isoflavones) are polyphenols present in many foods of plant origin including citrus fruits, green tea, red wine, and cocoa (Harnly et al., J Agric Food Chem, 54:9966-77, 2006). Large epidemiological studies link increased consumption of flavonoid-rich foods with reduced cardiovascular morbidity and mortality (Hertog et al., Lancet, 342:1007-11, 1993; Hertog et al., Arch Intern Med, 155:381-6, 1995; Mink et al., Am J Clin Nutr, 85:895-909, 2007). However, molecular and physiological mechanisms underlying potential cardiovascular health benefits of flavonoid consumption are poorly understood.

Flavanone glycosides hesperidin and naringin are present in citrus fruits. Hesperidin (hesperetin-7-O-rutinoside) is deglycosylated by intestinal microflora in the colon to produce the active aglycone hesperetin, that is then absorbed in the gut and subsequently glucuronidated to hesperetin glucuronide that circulates in plasma (Williamson et al., Am J Clin Nutr, 81:243 S-255S, 2005; Nielsen et al., J Nutr, 136:404-8, 2006). Hesperidin and naringin may have anti-inflammatory, hypolipidemic, and vasoprotective properties (Choe et al., J Cardiovasc Pharmacol, 38:947-55, 2001; Kim et al., Aging Cell, 5:401-11, 2006; et al., J Nutr Sci Vitaminol (Tokyo), 50:211-8, 2004; Jung et al., Clin Nutr, 22:561-8, 2003; Ohtsuki et al., J Nutr Sci Vitaminol (Tokyo), 48:420-2, 2002). Similar to insulin, naringenin (the aglycone of naringin) reduces apolipoprotein B100 secretion in HepG2 hepatoma cells by activating both phosphatidylinositol 3-kinase (PI3K)- and mitogen activated protein kinase (MAPK)-dependent signaling pathways (Allister et al., Diabetes, 54:1676-83, 2005; Borradaile et al., Diabetes, 52:2554-61, 2003; Borradaile et al., Lipids, 34:591-8, 1999).

Dyslipidemias contribute to endothelial dysfunction and accelerated atherosclerosis, in part, by promoting imbalances between endothelial-derived vasoconstrictors and vasodilators, growth factors, and pro- and anti-coagulant factors (Davignon & Ganz, Circulation, 109:III-27-III-32, 2004). Endothelial dysfunction often manifests as impaired endothelium-dependent vasodilator actions secondary to decreased production and/or bioavailability of nitric oxide (NO). It has recently been demonstrated that the green tea polyphenol epigallocatechin gallate (EGCG, a flavan-3-ol) acutely stimulates production of NO from vascular endothelium by activating signaling pathways involving Fyn/PI3K/Akt/eNOS (Kim et al., J Biol Chem, 282:13736-45, 2007). Moreover, three week treatment of Spontaneously Hypertensive Rats (SHR, a model of human metabolic syndrome) with EGCG lowers blood pressure, improves endothelial dysfunction and insulin resistance, and protects against myocardial ischemia/reperfusion injury (Potenza et al., Am J Physiol Endocrinol Metab, 292:E1378-87, 2007). In human studies, EGCG or polyphenol-rich cocoa intake improves endothelial dysfunction (Hooper et al., Am J Clin Nutr, 88:38-50, 2008; Muniyappa et al., Am J Clin Nutr, 88:1685-96, 2008; Widlansky et al., J Am Coll Nutr, 26:95-102, 2007).

### SUMMARY

Disclosed herein are methods of treating metabolic syndrome or insulin resistance. The methods include administering a therapeutically effective amount of purified hesperidin, purified hesperetin, or a purified derivative or analog of either compound to a subject with metabolic syndrome or insulin resistance (alone or in the context of metabolic syndrome). In some examples, the method includes administering purified hesperidin, purified hesperetin, or a purified derivative or analog of either compound, with a purity of at least 75% (such as at least 80%, 90%, 95%, or more) by weight to a subject to treat metabolic syndrome and/or insulin resistance. The disclosed compounds can be administered in combination with a pharmaceutically acceptable carrier. In some examples, the purified hesperidin, purified hesperetin, or a purified derivative or analog of either compound is administered orally.

In some embodiments, administration of a therapeutically effective amount of purified hesperidin, purified hesperetin, or a purified derivative or analog of either compound treats metabolic syndrome by decreasing triglyceride levels, increasing high density lipoprotein levels, decreasing blood pressure, decreasing blood glucose or insulin levels, decreasing levels of markers of prothrombotic or proinflammatory states, decreasing levels of vascular adhesion molecules, or a combination of two or more thereof as compared to a control or reference level. In other embodiments, administration of a therapeutically effective amount of purified hesperidin, purified hesperetin, or a purified derivative or analog of either compound treats insulin resistance by decreasing blood glucose levels, decreasing blood insulin levels, decreasing insulin sensitivity index, decreasing homeostatic model assessment score, decreasing quantitative insulin sensitivity check index score, or a combination of two or more thereof as compared to a control or reference level.

The foregoing and other features and advantages of the disclosure will become more apparent from the following detailed description of several embodiments, which proceeds with reference to the accompanying figures.

### BRIEF DESCRIPTION OF THE FIGURES

FIG. 1A is a digital image (upper) and a graph (lower) showing phosphorylated levels of AMP-activated protein kinase (AMPK), Akt, and endothelial nitric oxide synthase (eNOS) in bovine aortic endothelial cells (BAEC) that were serum-starved overnight and then treated with hesperetin for 10 minutes at the indicated concentrations. Cell lysates were immunoblotted with anti-phospho-AMPK (Thr172), anti-phospho-Akt (Ser473), anti-phospho-eNOS (Ser1179), and anti-β-actin (loading control) antibodies. Representative blots are shown (upper) for experiments that were repeated independently 5-6 times. Scanning densitometry was used to quantify results of multiple independent experiments (mean±SEM) (lower). Significant concentration-dependent effects of hesperetin to increase pAMPK, pAkt, and peNOS were observed (p<0.05; one-way ANOVA; *p<0.05 for Dunnett's comparisons to control (no hesperetin) for pAMPK, pAkt, and peNOS.

FIG. 1B is a digital image (upper) and a graph (lower) showing phosphorylated levels of AMPK, Akt, and eNOS in BAECs that were serum-starved overnight and then treated with hesperetin (10 μM) for the indicated durations. Cell lysates were immunoblotted as in FIG. 1A. Representative blots are shown for experiments that were repeated independently 5-6 times (upper). Scanning densitometry was used to quantify results of multiple independent experiments (mean±SEM) (lower). Significant time-dependent effects of hesperetin to increase pAMPK, pAkt, and peNOS were observed (p<0.05; one-way ANOVA); *p<0.05 for Dunnett's comparisons to control (0 min) for pAMPK, pAkt, or peNOS.

FIG. 1C is a series of digital images showing NO production in cells treated with hesperetin. BAECs were loaded with 4,5-diaminofluorescein diacetate (DAF-2-DA) prior to treatment with hesperetin for 10 minutes at the indicated concentrations (upper) or with hesperetin (10 μM) for the indicated durations (lower). Cells were fixed and observed. Emission of green light (510 nm) from cells excited by light at 480 nm is indicative of NO production. Phase contrast views of cells are also shown. A representative experiment is shown for experiments that were repeated independently three times.

FIG. 1D is a series of digital images showing NO production in cells subjected to various treatments. BAECs prepared as in FIG. 1C were treated without or with insulin (100 nM, 5 minutes), hesperetin (10 μM, 10 minutes), or lysophosphatidic acid (LPA, 5 μM, 5 minutes). In some groups of cells, the NO synthase inhibitor L-N^{G}-Nitroarginine methyl ester (L-NAME; 100 μM), the PI3K inhibitor Wortmannin (100 nM), or the AMPK inhibitor Compound C was added 30 minutes before loading cells with DAF-2-DA. A representative set of experiments is shown for experiments that were repeated independently five times.

FIG. 2A is a series of digital images showing H₂O₂ production in BAEC cells. BAEC were serum starved overnight and then loaded with 5-(and -6)-chloromethyl-2,7-dichlorodihydrofluorescein diacetate (CM-H₂DCF-DA). Cells were then treated with H₂O₂ (1 μM, 5 minutes), hesperetin (10 μM, 10 minutes), or naringenin (Nar, 10 μM, 10 minutes). In some groups of cells, N-acetylcysteine (NAC, 10 mM) was added 2 hours before loading with CM-H₂DCF-DA, to scavenge reactive oxygen species. After treatments, cells were viewed using an epifluorescent microscope. Emission of green light (510 nm) from cells excited by light at 480 nm is indicative of H₂O₂ production. A representative set of experiments is shown for experiments that were repeated independently three times.

FIG. 2B is a series of digital images of immunoblots. BAEC were serum-starved overnight and then treated with hesperetin (10 μM, 10 minutes) or naringenin (10 μM, 10 minutes). Some groups of cells were pre-treated with NAC (10 mM) for 2 hours prior to hesperetin or naringenin treatment. Cell lysates were then subjected to immunoblotting with the indicated antibodies. Immunoblots were obtained from gels run in parallel and probed with anti-phospho-src (Tyr418), anti-phospho-eNOS (Ser1179), anti-phospho-Akt (Ser473), anti-phospho-AMPK (Thr172), and anti-Akt antibodies. Representative blots are shown from experiments that were repeated independently three times.

FIG. 3A is series of digital images of immunoblots. BAECs were serum-starved overnight and then treated without or with EGCG (10 μM, 15 minutes), hesperetin (10 μM, 10 minutes), or naringenin (10 μM, 10 minutes). Some groups of cells were pre-treated with the src-family kinase inhibitor PP2 (1 μM, 1 hour). Cell lysates were subjected to immunoblotting with the indicated antibodies. Immunoblots were obtained from gels run in parallel and immunoblotted with anti-phospho-src (Tyr418), anti-phospho-Akt (Ser473), anti-phospho-eNOS (Ser1179), or anti-Akt antibodies (loading control). Representative blots are shown from experiments that were repeated independently three times.

FIG. 3B is a series of digital images showing NO production in BAEC cells. BAECs were serum-starved overnight and loaded with DAF-2 DA prior to treatment without or with insulin (100 nM, 5 minutes), EGCG (10 μM, 15 minutes), hesperetin (10 μM, 10 minutes) or naringenin (10 μM, 10 minutes). In some groups of cells the src family kinase inhibitor PP2 (1 μM) was added 30 minutes before loading cells with DAF-2 DA. After treatments, cells were fixed in 4% paraformaldehyde for 5 minutes at 4° C. and then viewed using an epifluorescent microscope. Emission of green light (510 nm) from cells excited by light at 480 nm is indicative of NO production. A representative set of experiments is shown for experiments that were repeated independently three times.

FIG. 4 is a schematic diagram depicting signaling pathways believed to be used by hesperetin and naringenin to stimulate production of NO in vascular endothelial cells.

FIG. 5A is a digital image showing vascular cell adhesion molecule-1 (VCAM-1) expression by immunoblotting. BAECs were serum-starved overnight and then treated without or with TNF-α (10 ng/ml, 5 hours). In some groups, cells were pre-treated with hesperetin (10 μM, 1 hour) prior to treatment with TNF-α. Cell lysates were immunoblotted using antibodies against VCAM-1 or β-actin. Representative immunoblots are shown for experiments that were repeated independently four times.

FIG. 5B is a graph showing results of scanning densitometry used to quantify results of multiple independent experiments represented in FIG. 5A (mean±SEM; n=4). Bars labeled with different letters are significantly different from each other. *p<0.001, by one-way ANOVA and Bonfferoni's post-test.

FIG. 5C is a series of digital images showing adherent monocytes in culture. BAECs were treated without or with TNF-α in the absence or presence of pre-treatment with hesperetin as in FIG. 5A. Then calcein-AM-labeled U937 monocytes were co-cultured with the treated BAEC. Monocytes adhering to the BAECs were visualized using an epifluorescent microscope (upper panel). Phase contrast view of the cells is shown in the lower panel. A representative set of experiments is shown for experiments that were repeated independently five times.

FIG. 6 is a schematic diagram of participant flow through the clinical trial described in Example 2.

FIG. 7A is a chromatogram of normal plasma.

FIG. 7B is a chromatogram of normal plasma spiked with hesperetin (300 ng/ml) and an internal standard.

FIG. 8A is a graph showing a calibration curve of calibration standard samples (plasma spiked with hesperetin and internal standard).

FIG. 8B is a graph showing a calibration curve of working solutions of hesperetin.

### DETAILED DESCRIPTION

### I. Abbreviations

apo B apolipoprotein B

BAEC bovine aortic endothelial cells

CM-H₂DCF-DA 5-(and -6)-chloromethyl-2,7-dichlorodihydrofluorescein diacetate

CRP C-reactive protein

DAF-2 DA 4,5-diaminofluorescein diacetate

EBM endothelial basal medium

EGCG epigallocatechin gallate

eNOS endothelial nitric oxide synthase

FMD flow-mediated dilation

FPG fasting plasma glucose

FSIVGTT frequently sampled intravenous glucose tolerance test

HDL high density lipoprotein

HOMA homeostatic model assessment

ICAM intracellular adhesion molecule

IGT impaired glucose tolerance

LDL low density lipoprotein

MCP-1 monocyte chemoattractant protein 1

NAC N-acetylcysteine

NMD nitroglycerin-mediated dilation

NO nitric oxide

OGTT oral glucose tolerance test

PAI-1 plasminogen activator inhibitor-1

PI3K phosphoinositide 3-kinase

PVP polyvinylpyrrolidone

QUICKI quantitative insulin-sensitivity check index

ROS reactive oxygen species

SAA serum amyloid A

TG triglycerides

VCAM vascular cell adhesion molecule

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Control: A “control” refers to a sample or standard used for comparison with an experimental sample. In some embodiments, the control is a sample obtained from a healthy subject (such as a subject without metabolic syndrome or insulin resistance or a non-obese subject). In some embodiments, the control is a historical control or standard reference value or range of values (such as a previously tested control sample, such as a group of subjects with metabolic syndrome or insulin resistance, or group of samples from subjects that do not have metabolic syndrome or insulin resistance). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval).

Derivative: A compound or portion of a compound that is derived from or is theoretically derivable from a parent compound (such as hesperidin or hesperetin), for example if at least one atom is replaced with another atom or group of atoms. Derivatives also include compounds to which at least one atom or functional group is added or removed, rather than replacing an atom or functional group of the parent compound.

In one example, a derivative of hesperidin includes hesperetin-7-glucoside.

Flavonoid: Polyphenol compounds, including flavones, flavonols, flavanones, and isoflavones, which are present in many foods of plant origin, such as citrus fruits, green tea, red wine, and cocoa. Consumption of flavonoid-rich foods has been linked with reduced cardiovascular morbidity and mortality. Examples of flavonoids found in foods include hesperidin, naringin, quercetin, and epigallocatechin gallate. Flavonoids also include metabolites of the flavonoids found in foods, for example hesperetin and naringenin.

Hesperidin (hesperetin-7-O-rutinoside): A flavanone glycoside that is present in citrus fruits (such as orange and lemon) having the structure:

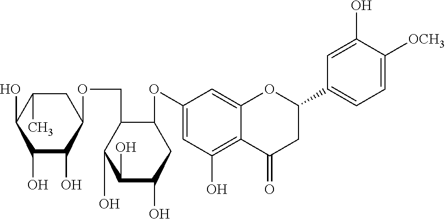

Hesperidin is deglycosylated by intestinal microflora in the colon to produce the active aglycone hesperetin, which is absorbed by the gut. Hesperetin has the structure:

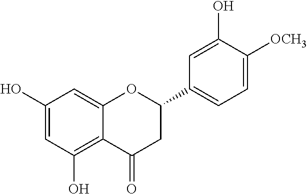 [SMILES: COc1ccc([C@@H]2CC(=O)c3c(O)cc(O)cc3O2)cc1O]

Insulin resistance: A state in which the cells of a subject do not respond appropriately to insulin, and increased amounts of insulin are required for glucose to be taken up by the cells. In some examples, insulin resistance is defined as a state where 200 units of insulin per day or more are required to attain glycemic control and prevent ketosis. Subjects with insulin resistance often have increased plasma glucose levels, increased plasma insulin levels, or both, as compared with a subject without insulin resistance or standard normal ranges.

In some examples, insulin resistance is determined by measuring blood glucose (such as fasting plasma glucose) and/or blood insulin (such as fasting plasma insulin) levels. In other examples, insulin resistance is determined by oral glucose tolerance test, glucose clamp (such as hyperinsulinemic euglycemic clamp), modified insulin suppression test, homeostatic model assessment, or quantitative insulin sensitivity check index (QUICKI).

Isolated: An isolated biological component (such as a nucleic acid, protein, or a compound, such as a flavonoid) has been substantially separated, produced apart from, or purified away from other biological components in the cell of the organism in which the component naturally occurs, for example the separation of a peptide from a sample, such as saliva, urine, serum or blood. An isolated compound (such as a naturally occurring plant compound, for example a flavonoid) has been substantially separated, produced apart from, or purified away from other compounds and biological components of the cell or organism in which it occurs. Flavonoids (such as hesperidin or hesperetin) that have been isolated include flavonoids purified by standard purification methods, such as chromatography, for example high performance liquid chromatography (HPLC) and the like. The term also embraces proteins and compounds prepared by recombinant expression in a host cell, as well as chemically synthesized peptides, nucleic acids, and other compounds (such as flavonoids, for example, hesperidin or hesperetin). It is understood that the term “isolated” does not imply that the biological component is free of trace contamination, and can include molecules that are at least 50% isolated, such as at least 50%, 55%, 60%, 65%, 70%, 75%, 80%, 85%, 90%, 95%, 98%, 99%, or even 100% isolated.

Metabolic syndrome: A group of metabolic risk factors, including abdominal obesity, atherogenic dyslipidemia, elevated blood pressure, insulin resistance, pro-thrombotic state, and pro-inflammatory state, that correlate with increased risk of coronary heart disease, stroke, and type 2 diabetes. As much as 20-25% of the adult population worldwide is estimated to have metabolic syndrome. Treatment of metabolic syndrome currently includes lifestyle changes (including weight loss and increased physical activity). If lifestyle changes are not effective, drug therapy for individual components of the metabolic syndrome (such as cholesterol lowering drugs and anti-hypertensives) are often administered.

There are a number of different guidelines for diagnosing metabolic syndrome. In one example, metabolic syndrome is diagnosed using the National Cholesterol Education Program Adult Treatment Panel 111 (NCEP ATPIII) criteria, which include at least three of: central obesity (waist circumference≧40 inches (male) or ≧36 inches (female)), triglycerides≧1.7 mM (150 mg/dL), high density lipoprotein (HDL)<40 mg/dL (male) or <50 mg/dL (female), blood pressure≧130/85 mm Hg, and fasting plasma glucose≧6.1 mM (110 mg/dL). In another example, metabolic syndrome is diagnosed using the International Diabetes Federation criteria, which include central obesity (waist circumference based on ethnic specific values or body mass index>30 kg/m²) and any two of: triglycerides of ≧150 mg/dL or specific treatment for this lipid abnormality, HDL<40 mg/dL (male) or <50 mg/dL (female) or specific treatment for this lipid abnormality, systolic blood pressure≧135 mm Hg or diastolic blood pressure≧85 mm Hg or treatment of previously diagnosed hypertension, and fasting plasma glucose≧100 mg/dL or previously diagnosed type 2 diabetes. See, e.g., Expert Panel on Detection, Evaluation, and Treatment of High Blood Cholesterol in Adults, J. Am. Med. Assn. 285:2486-2497, 2001; Alberti et al., Diabet. Med. 23:469-480, 2006.

Obesity: A condition in which excess body fat may put a person at health risk (see Barlow and Dietz, Pediatrics 102: E29, 1998; National Institutes of Health, Obes. Res. 6 (suppl. 2):51S-209S, 1998). Excess body fat is a result of an imbalance of energy intake and energy expenditure. Body Mass Index (BMI) can be used to assess obesity. BMI is calculated by dividing weight (in kg) by height² (in meters²). Grade I obesity (also called “overweight”) corresponds to a BMI of 25-29.9 kg/m². Grade II obesity corresponds to a BMI of 30-40 kg/m²; and Grade III obesity corresponds to a BMI greater than 40 kg/m² (Jequier, Am. J. Clin. Nutr., 45:1035-47, 1987). Waist circumference is another measurement used to assess obesity. In men, a waist circumference of 102 cm (40 inches) or more is considered obese, while in women a waist circumference of 89 cm (35 inches) or more is considered obese.

Pharmaceutically acceptable carrier: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of compounds, such as hesperidin, hesperetin, or a derivative or analog of either compound.

In general, the nature of the carrier will depend on the particular mode of administration employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate.

Purified: The term purified does not require absolute purity; rather, it is intended as a relative term. Thus, for example, a purified hesperidin or hesperetin preparation is one in which the hesperidin or hesperetin is more enriched than in its natural environment within a cell or a plant product (such as citrus fruit, for example, orange or lemon peel). Preferably, a preparation is purified such that the hesperidin or hesperetin represents at least 50% of the total content of the preparation, for example, at least 50% by weight.

Substantially purified hesperidin or hesperetin as used herein refers to hesperidin or hesperetin that is substantially free of proteins, lipids, carbohydrates or other materials with which it is naturally associated. In one embodiment, the hesperidin or hesperetin is at least 50%, for example at least 70%, at least 75%, at least 80%, at least 85%, at least 90%, at least 95%, or more free of proteins, lipids, carbohydrates or other materials with which it is naturally associated.

Subject: A living multi-cellular vertebrate organism, a category that includes both human and non-human mammals.

Therapeutically effective amount: An amount or dose sufficient to prevent advancement, or to cause regression of a disease or syndrome or is capable of relieving symptoms of a disease or syndrome (such as metabolic syndrome or insulin resistance).

Treating or inhibiting a disease: “Inhibiting” a disease refers to inhibiting the full development of a disease or disorder, for example in a person who is known to have a disorder such as metabolic syndrome or insulin resistance or a predisposition to such a disorder. Inhibition of a disorder can span the spectrum from partial inhibition to substantially complete inhibition (prevention) of the disorder. In some examples, the term “inhibiting” refers to reducing or delaying the onset or progression of a disorder. A subject to be administered with a therapeutically effective amount of the pharmaceutical compound to inhibit or treat the disorder can be identified by standard diagnosing techniques for such a disorder, for example, basis of clinical symptoms, family history, or risk factor to develop the disease or disorder. “Treatment” refers to a therapeutic intervention that ameliorates a sign or symptom of a disease or disorder after it has begun to develop.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Disclosed herein are methods for treating metabolic syndrome and/or insulin resistance utilizing the citrus polyphenol hesperidin and its aglycone form, hesperetin. In some examples, the method includes administering a therapeutically effective amount of purified hesperetin (for example, hesperetin having a purity of at least 75%) to a subject, for example a subject with metabolic syndrome (which may include insulin resistance) or a subject with insulin resistance in the absence of metabolic syndrome. In other examples, the method includes administering a therapeutically effective amount of purified hesperidin (for example, hesperidin having a purity of at least 75%) to a subject, for example, a subject with metabolic syndrome (which may include insulin resistance) or a subject with insulin resistance in the absence of metabolic syndrome. In further examples, the method includes administering a therapeutically effective amount of a purified derivative of hesperidin or hesperetin (such as hesperetin-7-glucoside) to a subject with metabolic syndrome or insulin resistance.

In some embodiments, treating metabolic syndrome includes treating one or more symptoms of metabolic syndrome, such as decreasing triglyceride levels, increasing high density lipoprotein (HDL) levels, decreasing blood pressure, decreasing fasting blood glucose, or a combination of two or more thereof in the subject as compared with a control. In other embodiments, treating insulin resistance includes treating one or more symptoms of insulin resistance, for example, decreasing plasma glucose (such as fasting plasma glucose) or decreasing plasma insulin (such as fasting plasma insulin) in the subject as compared to a control.

The disclosed methods include administering purified hesperidin, purified hesperetin, or purified derivatives or analogs of either compound (such as hesperetin-7-glucoside) to a subject. In some examples, the methods include administering a composition including about 1 mg/kg to about 3 mg/kg purified hesperidin to the subject. In particular examples, the agent is administered orally. In some examples, the purified hesperidin, purified hesperetin, or a purified derivative or analog of either compound is included in a composition that includes a pharmaceutically acceptable carrier. In one example, the composition consists essentially of purified hesperetin and a pharmaceutically acceptable carrier. In another example, the composition consists essentially of purified hesperidin and a pharmaceutically acceptable carrier.

### Iv. Methods of Treating Metabolic Syndrome and/or Insulin Resistance

Disclosed herein are methods of treating metabolic syndrome and/or insulin resistance by administering a therapeutically effective amount of purified hesperidin, purified hesperetin, or purified derivatives or analogs of either compound to a subject with metabolic syndrome or insulin resistance.

In particular embodiments, the methods include selecting a subject for treatment with the methods and compositions disclosed herein. In some examples, the methods include selecting a subject with obesity and one or more early cardiovascular symptoms (including, but not limited to increased blood pressure, increased atherogenic markers, increased circulating inflammatory markers, increased cell adhesion markers, or a history of heart attack) for treatment. In some examples, the selected subject also has insulin resistance. In some examples, the selected subject is obese, for example, has a BMI of 25 kg/m² or more (for example at least 25, 26, 27, 28, 29, 30, 31, 32, 33, 34, 35, 36, 37, 38, 39, 40, 41, 42, 43, 44, 45 kg/m², or more) and/or a waist circumference of 102 cm or more (men) or a waist circumference of 89 cm or more (women). The selected subject also has one or more of increased blood pressure, increased atherogenic markers (such as plasminogen activator inhibitor-1 (PAI-1), monocyte chemoattractant protein 1 (MCP-1), fibrinogen, and/or serum amyloid A (SAA) protein), increased circulating inflammatory markers (such as C-reactive protein (CRP), SAA protein, and/or homocysteine), increased cell adhesion markers (such as vascular cell adhesion molecule 1 (VCAM-1), intracellular adhesion molecule 1 (ICAM-1), and/or E-selectin), or a history of heart attack (for example, one or more past heart attacks). In some examples, blood pressure, atherogenic markers, inflammatory markers, and or cell adhesion markers are increased as compared to a control (such as a non-obese subject or population). In some examples, a subject is identified as having increased blood pressure if the subject's blood pressure is ≧130/85 mm Hg, systolic blood pressure≧135 mm Hg or diastolic blood pressure≧85 mm Hg, or if the subject has or had treatment of previously diagnosed hypertension. In further non-limiting examples, the selected subject has PAI-1 levels greater than about 10 ng/ml (such as greater than about 10 ng/ml, 15 ng/ml, 20 ng/ml, 25 ng/ml, 30 ng/ml, or more), MCP-1 levels greater than about 100 pg/ml (such as greater than about 100 pg/ml, 150 pg/ml, 200 pg/ml, 250 pg/ml, or more), fibrinogen levels greater than about 300 mg/dL (such as greater than about 300 mg/dL, 325 mg/dL, 350 mg/dL, 375 mg/dL, 400 mg/dL, 425 mg/dL, 450 mg/dL, or more), and/or SAA levels greater than about 5 mg/L (such as greater than about 5 mg/L, 10 mg/L, 15 mg/L, 20 mg/L, or more). In other non-limiting examples, homocysteine levels are greater than about 10 μM (such as greater than about 10 μM, 15 μM, 20 μM, 25 μM, 30 μM, or more), CRP levels are greater than about 2.0 mg/L (such as greater than about 2.0 mg/L, 2.5 mg/L, 3.0 mg/L, 3.5 mg/L or more), and/or SAA levels are greater than about 5 mg/L (such as greater than about 5 mg/L, 10 mg/L, 15 mg/L, 20 mg/L, or more) in the selected subject. In additional non-limiting examples, VCAM-1 levels are greater than about 500 ng/ml (such as greater than about 500 ng/ml, 600 ng/ml, 700 ng/ml, 800 ng/ml, 900 ng/ml, or more), ICAM-1 levels are greater than about 150 ng/ml (such as greater than about 150 ng/ml, 200 ng/ml, 250 ng/ml, 300 ng/ml, or more), and/or E-selectin levels are greater than about 30 ng/ml (such as greater than about 30 ng/ml, 35 ng/ml, 40 ng/ml, 45 ng/ml, 50 ng/ml, or more) in the selected subject.

### A. Metabolic Syndrome

In some embodiments, the disclosed methods include treating a subject with metabolic syndrome. Metabolic syndrome (sometimes referred to as Syndrome X, insulin resistance syndrome, or CHAOS) is a group of metabolic risk factors, including abdominal obesity, atherogenic dyslipidemia, elevated blood pressure, insulin resistance, prothrombotic state, and proinflammatory state, which correlate with increased risk of coronary heart disease, stroke, and type 2 diabetes. As much as 20-25% of the adult population worldwide is estimated to have metabolic syndrome.

There are a number of different guidelines for diagnosing metabolic syndrome. In one example, metabolic syndrome is diagnosed using the National Cholesterol Education Program Adult Treatment Panel 111 (NCEP ATPIII) criteria, which include at least three of central obesity (waist circumference≧40 inches (male) or ≧36 inches (female)), triglycerides≧1.7 mM (150 mg/dL), high density lipoprotein (HDL)<40 mg/dL (male) or <50 mg/dL (female), blood pressure≧130/85 mm Hg, and fasting plasma glucose≧6.1 mM (110 mg/dL). In another example, metabolic syndrome is diagnosed using the International Diabetes Federation criteria, which include central obesity (waist circumference based on ethnic specific values or body mass index>30 kg/m²) and any two of triglycerides of ≧150 mg/dL or specific treatment for this lipid abnormality, HDL<40 mg/dL (male) or <50 mg/dL (female) or specific treatment for this lipid abnormality, systolic blood pressure≧135 mm Hg or diastolic blood pressure≧85 mm Hg or treatment of previously diagnosed hypertension, and fasting plasma glucose≧100 mg/dL or previously diagnosed type 2 diabetes. See, e.g., Expert Panel on Detection, Evaluation, and Treatment of High Blood Cholesterol in Adults, J. Am. Med. Assn. 285:2486-2497, 2001; Alberti et al., Diabet. Med. 23:469-480, 2006. Other criteria for diagnosing metabolic syndrome include those from the European Group for the Study of Insulin Resistance, the World Health Organization, and the American Heart Association (e.g., Grundy et al., Circulation 109:433-438, 2004; Alberti et al., Diabet. Med. 15:539-553, 1998; Balkau and Charles, Diabet. Med. 16:442-443, 1999). One of skill in the art can utilize one or more of these guidelines to identify a subject with metabolic syndrome or at risk of developing metabolic syndrome.

In some embodiments, the disclosed methods include treating metabolic syndrome by decreasing triglyceride levels, increasing HDL levels, decreasing blood pressure, decreasing fasting blood glucose, decreasing levels of markers of a prothrombotic state, decreasing levels of markers of a proinflammatory state, or decreasing levels of vascular adhesion molecules in a subject as compared with a control. In some examples, the method includes two or more (such as 3, 4, 5, 6, or 7) of decreasing triglyceride levels, increasing HDL levels, decreasing blood pressure, decreasing fasting blood glucose, decreasing levels of markers of a prothrombotic state, decreasing levels of markers of a proinflammatory state, or decreasing levels of vascular adhesion molecules in a subject as compared with a control.

In some examples, administration of purified hesperidin or purified hesperetin or a purified derivative or analog of either compound treats metabolic syndrome by decreasing triglyceride levels in a subject, for example decreasing triglyceride levels by at least 5% (such as at least 10%, 15%, 20%, 25%, 30%, 35%, or more) as compared with a control. In some examples, the method includes decreasing triglyceride levels in a subject to <150 mg/dL. Methods of determining triglyceride levels in a subject (for example in a blood sample from a subject) are routine. In further examples, administration of purified hesperidin or purified hesperetin or a purified derivative or analog of either compound treats metabolic syndrome by increasing HDL levels in a subject, for example, increasing HDL levels by at least 5% (such as at least 10%, 15%, 20%, 25%, 30%, 35%, or more) as compared with a control. In some examples, the method includes increasing HDL levels in a male subject to ≧40 mg/dL or increasing HDL levels in a female subject to ≧50 mg/dL. Methods of determining HDL levels in a subject (for example, in a blood sample from a subject) are routine.

In additional examples, administration of purified hesperidin or purified hesperetin or a purified derivative or analog of either compound treats metabolic syndrome by decreasing blood pressure in a subject, for example, decreasing blood pressure (such as systolic pressure, diastolic pressure, or both) by at least 5% (such as at least 10%, 15%, 20%, 25%, 30%, 35%, or more) as compared with a control. In some examples, the method includes decreasing blood pressure in the subject to <135/85 mm Hg or decreasing systolic blood pressure to <135 mm Hg or diastolic blood pressure to <85 mm Hg.

In further examples, administration of purified hesperidin or purified hesperetin or a purified derivative or analog of either compound treats metabolic syndrome by decreasing blood glucose levels (such as fasting plasma glucose (FPG)) in a subject, for example, decreasing blood glucose by at least 5% (such as at least 10%, 15%, 20%, 25%, 30%, 35%, or more) as compared with a control. In some examples, the method includes decreasing FPG to <110 mg/dL. Methods to measure blood glucose levels in a subject (for example, in a blood sample from a subject) are routine.

In still further examples, administration of purified hesperidin or purified hesperetin or a purified derivative or analog of either compound treats metabolic syndrome by decreasing markers of a prothrombotic state (such as plasminogen activator inhibitor-1 (PAI-1) or fibrinogen) in a subject, for example, decreasing levels of PAI-1 and/or fibrinogen by at least 5% (such as at least 10%, 15%, 20%, 25%, 30%, 35%, or more) as compared with a control. In additional examples, administration of purified hesperidin or purified hesperetin or a purified derivative or analog of either compound treats metabolic syndrome by decreasing markers of a proinflammatory state (such as CRP, SAA protein, or homocysteine) in a subject, for example decreasing levels of CRP, SAA, and/or homocysteine by at least 5% (such as at least 10%, 15%, 20%, 25%, 30%, 35%, or more) as compared with a control. In further examples, administration of purified hesperidin or purified hesperetin or a purified derivative or analog of either compound treats metabolic syndrome by decreasing vascular adhesion molecules (such as vascular cell adhesion molecule (VCAM), intracellular adhesion molecule (ICAM), or E-selectin) in a subject, for example, decreasing levels of VCAM, ICAM, or E-selection by at least 5% (such as at least 10%, 15%, 20%, 25%, 30%, 35%, or more) as compared with a control. Levels of prothrombotic, proinflammatory, or vascular adhesion molecules in a subject (for example, in a blood sample from a subject) can be determined by routine methods, such as immunoassay (for example, by ELISA).

### B. Insulin Resistance

In some embodiments, the disclosed methods include treating a subject with insulin resistance. In some examples, a subject with insulin resistance is a subject with metabolic syndrome, while in other examples, a subject with insulin resistance does not have metabolic syndrome, but may, for example, be pre-diabetic. Insulin resistance is a decreased sensitivity or responsiveness to the metabolic actions of insulin. In some examples, insulin resistance results in increased blood glucose and/or increased blood insulin levels (such as fasting blood glucose or fasting blood insulin levels).

In some examples, insulin resistance is determined by hyperinsulinemic euglycemic clamp (glucose clamp), which measures the amount of glucose necessary to compensate for increased insulin levels without causing hypoglycemia (see, e.g., DeFronzo et al., Am. J. Physiol. 237:E214-E223, 1979). In one example, the glucose clamp method includes infusing insulin in a subject at 10-120 mU/m²/min and infusing 20% glucose to maintain blood glucose levels between about 90-100 mg/dL. If low levels of glucose (such as ≦4 mg/min) are required to maintain blood glucose levels, then the subject is considered insulin resistant. High levels of glucose (such as ≧7.5 mg/min) indicate that the subject is insulin sensitive, while between 4-7.5 mg/min of glucose is considered to indicate impaired glucose tolerance (IGT), which is an early sign of insulin resistance.

In some examples of the disclosed method, administration of purified hesperidin or purified hesperetin or a purified derivative or analog of either compound treats insulin resistance by increasing the amount of glucose required to maintain blood glucose levels in a glucose clamp in a subject, for example, by at least 5% (such as at least 10%, 15%, 20%, 25%, 30%, 35%, or more) as compared with a control. In some examples, the method includes increasing the amount of glucose required to maintain blood glucose levels in a glucose clamp to ≧4 mg/min glucose. In other examples, the method includes increasing the amount of glucose required to maintain blood glucose levels in a glucose clamp to ≧7.5 mg/min glucose.

In another example, insulin resistance is determined by the frequently sampled intravenous glucose tolerance test (FSIVGTT; Bergman, Diabetes 38:1512-1527, 1989). FSIVGTT is performed by administering intravenous glucose with frequent blood sampling to determine glucose and insulin levels. Insulin is injected 20 minutes after the start of glucose administration. The insulin sensitivity index (SI), reflecting increase in fractional glucose disappearance per unit of insulin increase, is calculated. In some examples, an SI value of ≦2 μU/min/mL indicates insulin resistance. In some examples of the disclosed method, administration of purified hesperidin or purified hesperetin or a purified derivative or analog of either compound treats insulin resistance by increasing the insulin sensitivity index of a subject, for example, by at least 5% (such as at least 10%, 15%, 20%, 25%, 30%, 35%, or more) as compared with a control. In some examples, the method includes increasing the insulin sensitivity index to ≧2 μU/min/mL.

In other examples, insulin resistance is determined by quantitative insulin sensitivity check index (QUICKI; Katz et al., J. Clin. Endocrinol. Metab. 85:2402-2410, 2000). QUICKI is calculated from fasting glucose and fasting insulin levels:

QUICKI=1/[(log(I₀)+(log(G₀)]

wherein I₀ is the fasting plasma insulin level (μU/mL) and G₀ is the fasting blood glucose level (mg/dL). In some examples of the disclosed method, administration of purified hesperidin or purified hesperetin or a purified derivative or analog of either compound treats insulin resistance by increasing the QUICKI value in a subject by at least 5% (such as at least 10%, 15%, 20%, 25%, 30%, 35%, or more) as compared with a control. In some examples, the method includes increasing the subject's QUICKI to >0.350.

In other examples, insulin resistance is determined by the homeostasis model assessment (HOMA-IR; Matthews et al., Diabetologia 28:412-429, 1985). HOMA-IR is calculated from fasting glucose and fasting insulin levels:

HOMA-IR=[fasting plasma insulin×fasting plasma glucose]/22.5

wherein fasting plasma insulin is expressed as μU/mL and fasting plasma glucose is expressed as mM. In some examples of the disclosed method, administration of purified hesperidin or purified hesperetin or a purified derivative or analog of either compound treats insulin resistance by decreasing the HOMA-IR value in a subject by at least 5% (such as at least 10%, 15%, 20%, 25%, 30%, 35%, or more) as compared with a control. In some examples, the method includes decreasing HOMA-IR to ≦4.

In some examples, insulin resistance includes impaired glucose tolerance (IGT), alone or in combination with impaired fasting glucose regulation. An oral glucose tolerance test (OGTT) can be used to determine if a subject has IGT. An OGTT two-hour plasma glucose of greater than or equal to 140 mg/dL and less than 200 mg/dL (7.8-11.0 mM) is considered to be IGT.

In some examples, fasting plasma glucose (FPG) of greater than about 100 mg/dL and less than 126 mg/dL (5.6-6.9 mM) indicates that a subject has impaired fasting glucose regulation or insulin resistance. In some examples, administration of purified hesperidin or purified hesperetin or a purified derivative or analog of either compound treats insulin resistance by decreasing plasma glucose levels (such as FPG or 2-hour glucose levels following oral glucose tolerance test (OGTT)) in a subject, for example, decreasing plasma glucose levels by at least 5% (such as at least 10%, 15%, 20%, 25%, 30%, 35%, or more) as compared with a control. In some examples, the method includes decreasing FPG to <110 or <100 mg/dL. Methods to measure plasma glucose in a subject (for example, in a blood sample from a subject) are routine, for example utilizing the glucose oxidase method.

In additional examples, administration of purified hesperidin or purified hesperetin or a purified derivative or analog of either compound treats insulin resistance by decreasing plasma insulin levels (such as fasting plasma insulin or 2-hour insulin levels following OGTT) in a subject, for example, decreasing plasma insulin levels by at least 5% (such as at least 10%, 15%, 20%, 25%, 30%, 35%, or more) as compared with a control. In some examples, the method includes decreasing fasting plasma insulin levels to <15 μU/mL. Methods to measure plasma insulin in a subject (for example, in a blood sample from a subject), such as immunoassays, are routine.

One of skill in the art will recognize that because of a lack of standardized assays, interassay variability in insulin measurements can confound defining universal ranges for insulin resistance and insulin sensitivity. Therefore, in some examples, insulin sensitive subjects include the top 25^{th} percentile of insulin sensitive subjects in a given cohort where insulin levels are measured in the same central reference laboratory. Similarly, in some examples, insulin resistant subjects include the bottom 25^{th} percentile of insulin sensitive subjects in a given cohort where insulin levels are measured in the same central reference laboratory. In additional examples, impaired glucose tolerance can be defined according the results of an oral glucose tolerance test using guidelines that are published by the American Diabetes Association. See, e.g., Diabetes Care 33:S62-S69, 2010.

### C. Controls

In some embodiments, the disclosed methods include comparing one or more indicator of metabolic syndrome (such as triglyceride levels, HDL levels, blood pressure, blood glucose levels, or levels of one or more markers of a prothrombotic state, a proinflammatory state or vascular adhesion) to a control, wherein an increase or decrease in the particular indicator relative to the control (as discussed above) indicates effective treatment of the metabolic syndrome. In other embodiments, the disclosed methods include comparing one or more indicator of insulin resistance (such as blood glucose levels, blood insulin levels, insulin sensitivity index, HOMA-IR, or QUICKI) to a control, wherein an increase or decrease in the particular indicator relative to the control (as discussed above) indicates effective treatment of insulin resistance.

The control can be any suitable control against which to compare the indicator of metabolic syndrome or insulin resistance in a subject. In some embodiments, the control is a sample obtained from a healthy subject (such as a subject without metabolic syndrome or insulin resistance or a non-obese subject). In some embodiments, the control is a historical control or standard reference value or range of values (such as a previously tested control sample, such as a group of subjects with metabolic syndrome or insulin resistance, or group of samples from subjects that do not have metabolic syndrome or insulin resistance). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study

### D. Purified Hesperidin, Hesperetin and Related Compounds

The disclosed methods include administering a therapeutically effective amount of a purified citrus flavanone, such as hesperidin, hesperetin, or a derivative or analog of either compound (such as hesperetin-7-glucoside) to a subject. A purified hesperidin or purified hesperetin preparation is one in which the hesperidin or hesperetin is more enriched than in its natural environment within a cell or a plant product (such as citrus fruit, for example, orange or lemon peel). In some examples, a preparation is purified such that the hesperidin or hesperetin is substantially free of proteins, lipids, carbohydrates or other materials with which it is naturally associated (for example other flavonoid compounds). In one example, the hesperidin or hesperetin is at least 50%, for example at least 55%, 60%, 65%, 70%, 75%, 80%, 85%, 90%, 95%, 96%, 97%, 98%, 99% or more free of proteins, lipids, carbohydrates or other materials with which it is naturally associated. In some examples, the purity of the hesperidin or hesperetin is determined by the percentage of hesperidin or hesperetin in the preparation by weight. For example, the purified hesperidin or purified hesperetin is at least 50% pure by weight (for example at least 55%, 60%, 65%, 70%, 75%, 80%, 85%, 90%, 95%, 96%, 97%, 98%, 99% or more pure by weight). Methods of obtaining purified hesperidin, purified hesperetin, or purified derivatives or analogs of either compound are well known to one of skill in the art.

In some examples, purified hesperidin is obtained by extraction from plant products, for example, citrus fruit (such as orange or lemon peel). In some examples, hesperidin is obtained by alkaline extraction, followed by filtration and neutralization or adsorption. In other examples, hesperidin is obtained by organic extraction or ultrasound-assisted extraction. See, e.g., U.S. Pat. No. 2,421,061; Pritchett et al., J. Am. Chem. Soc. 68:2108, 1946; Ikan, Natural Products: A Laboratory Guide, Academic Press, 1991; Di Mauro et al., J. Agric. Food Chem. 47:4391-4397, 1999; Ma et al., Ultrasonics Sonochem. 15:227-232, 2008. Purified hesperidin is also commercially available (e.g., Cat. No. H5254, Sigma-Aldrich, St. Louis, Mo.; Cat. No. 123460050, Acros Organics, Geel, Belgium).

In other examples, purified hesperetin is obtained by deglycosylating hesperidin, for example by acid hydrolysis (see, e.g., U.S. Pat. No. 4,150,038; Seitz and Wingard, J. Agric. Food Chem. 26:278-280, 1978; Ikan, Natural Products: A Laboratory Guide, Academic Press, 1991). Purified hesperetin can also be prepared by total synthesis (Zemplen and Bognar, Chem. Ber. 75B:1043, 1942; Honohan et al., J. Agric. Food Chem. 24:906, 1976). Finally, purified hesperetin is commercially available (e.g., Cat. No. H4125, Sigma-Aldrich, St. Louis, Mo.; Cat. No. 10006084, Cayman Chemical, Ann Arbor, Mich.).

In further examples, the methods disclosed herein include administration of purified hesperetin-7-glucoside to a subject. Methods of preparing hesperetin-7-glucoside are known to one of skill in the art. For example, partial acid hydrolysis of hesperidin at high temperatures can by used to prepare hesperetin-7-glucoside (see, e.g., Grohmann et al., Carb. Res. 328:141-146, 2000). One of skill in the art can identify additional hesperidin or hesperetin derivatives or analogs and methods for preparing purified preparations of such compounds (see, e.g., U.S. Pat. No. 6,831,098).

In some non-limiting examples of the methods described herein, an amount of purified hesperidin or purified hesperetin or a purified derivative or analog of either compound sufficient to achieve a plasma concentration (such as peak plasma concentration (Cₘₐₓ)) of about 0.5-10 μM is administered to a subject. Methods for determining the amount of hesperetin in a sample (such as plasma) are well known to one of skill in the art. In some examples, the method includes chromatographic methods, such as high pressure liquid chromatography (HPLC). In other examples, the method includes spectroscopic methods (such as nuclear magnetic resonance, mass spectrometry, or UV spectroscopy). See, e.g., Kanaze et al., J. Chromatogr. B 801:363-367, 2004; Nielsen et al., J. Nutr. 136:404-408, 2006; Spanakis et al., Biomed. Chromatogr. 23:124-131, 2008.

### V. Pharmaceutical Compositions and Administration

Pharmaceutical compositions that include purified hesperidin or purified hesperetin or a purified derivative or analog of either compound (such as purified hesperetin-7-glucoside) can be formulated with an appropriate pharmaceutically acceptable carrier, depending upon the particular mode of administration chosen. In one example, the pharmaceutical composition includes purified hesperidin and a pharmaceutically acceptable carrier. In another example, the pharmaceutical composition includes purified hesperetin and a pharmaceutically acceptable carrier. In some examples, the pharmaceutical composition consists essentially of purified hesperidin or purified hesperetin and a pharmaceutically acceptable carrier.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually comprise injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, the purified hesperidin or purified hesperetin or a purified derivative or analog of either compound is included in a controlled release formulation, for example, a microencapsulated formulation. Various types of biodegradable and biocompatible polymers, methods can be used, and methods of encapsulating a variety of synthetic compounds, proteins and nucleic acids, have been well described in the art (see, for example, U.S. Pat. Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2^{nd} ed., CRC Press, 2006).

In other examples, the purified hesperidin or purified hesperetin or a purified derivative or analog of either compound is included in a nanodispersion system. Nanodispersion systems and methods for producing such nanodispersions are well known to one of skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and purified hesperetin (such as 80/20 w/w). In some examples, the nanodispersion is prepared using the solvent evaporation method. See, e.g., Kanaze et al., Drug Dev. Indus. Pharm. 36:292-301, 2010; Kanaze et al., J. Appl. Polymer Sci. 102:460-471, 2006.

In some examples, the purified hesperidin, purified hesperetin, or purified derivatives or analogs of either compound, include pharmaceutically acceptable salts of such compounds. “Pharmaceutically acceptable salts” of the presently disclosed compounds include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Description of suitable pharmaceutically acceptable salts can be found in Handbook of Pharmaceutical Salts, Properties, Selection and Use, Wiley VCH (2002).

In some examples, the pharmaceutical compositions disclosed herein comprise purified hesperidin, purified hesperetin, or a purified derivative or analog of either compound, and at least one pharmaceutically acceptable carrier. In other examples, the composition consists essentially of purified hesperidin, purified hesperetin, or a purified derivative or analog of either compound and at least one pharmaceutically acceptable carrier. In the present disclosure, “consists essentially of” indicates that additional active compounds (for example additional flavonoids) are not included in the composition, but that other inert agents (such as fillers, wetting agents, or the like) can be included, and “consists of” indicates that additional agents are not included in the composition.

The dosage form of the pharmaceutical composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutical compositions that include purified hesperidin, purified hesperetin, or a purified analog or derivative of either compound can be formulated in unit dosage form, suitable for individual administration of precise dosages. In one specific, non-limiting example, a unit dosage contains from about 1 mg to about 1 g of purified hesperidin or purified hesperetin (such as about 100 mg to about 900 mg, about 250 mg to about 750 mg, or about 400 mg to about 600 mg purified hesperidin or purified hesperetin, such as about 500 mg hesperidin or hesperetin). In some examples, a unit dosage includes about 50 mg, 100 mg, 150 mg, 200 mg, 250 mg, 300 mg, 350 mg, 400 mg, 450 mg, 500 mg, or more purified hesperidin or purified hesperetin. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The compounds of this disclosure can be administered to humans or other animals on whose tissues they are effective in various manners such as orally, intravenously, intramuscularly, intraperitoneally, intranasally, intradermally, intrathecally, subcutaneously, via inhalation or via suppository. In one non-limiting example, the composition is administered orally. The particular mode of administration and the dosage regimen will be selected by the attending clinician, taking into account the particulars of the case (e.g. the subject, the disease, the disease state involved, and whether the treatment is prophylactic). Treatment can involve daily or multi-daily doses of compound(s) over a period of a few days to months, or even years. In a particular example, treatment involves daily or twice daily doses of purified hesperidin or purified hesperetin.

In some examples, a therapeutically effective amount of a purified hesperidin or purified hesperetin or a derivative or analog of either compound is about 0.5 mg/kg to about 50 mg/kg (for example, about 1 mg/kg to about 25 mg/kg or about 1 mg/kg to about 10 mg/kg). In some examples, a therapeutically effective amount of purified hesperidin or purified hesperetin is about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, or more mg/kg. In a specific example, a therapeutically effective amount of a purified hesperidin or purified hesperetin or a purified derivative or analog of either compound is about 1.0 mg/kg to about 3.0 mg/kg (such as about 1, 1.1, 1.2, 1.3, 1.4, 1.5, 1.6, 1.7, 1.8, 1.9, 2.0, 2.1, 2.2, 2.3, 2.4, 2.5, 26., 2.7, 2.8, 2.9, or 3.0 mg/kg). In other examples, a therapeutically effective amount of a purified hesperidin or purified hesperetin or a purified derivative or analog of either compound is an amount sufficient to achieve a plasma concentration of about 0.5-10 μM (such as about 1 μM to about 5 μM, about 1 μM to about 3 μM, or about 1 μM to about 3 μM) hesperetin in the subject. In some examples, the plasma concentration is the peak plasma concentration (Cₘₐₓ) of hesperetin in the subject. One of skill in the art can determine an amount of purified hesperidin or purified hesperetin or a derivative or analog of either compound sufficient to achieve a desired plasma concentration (such as Cₘₐₓ) of hesperetin utilizing pharmacokinetic studies performed in animal and/or human subjects. See, e.g., Nielsen et al., J. Nutr. 136:404-408, 2006; Kanaze et al., Eur. J. Clin. Nutr. 61:472-477, 2007.

A therapeutically effective amount of a purified hesperidin or purified hesperetin or a derivative or analog of either compound can be the amount of a purified hesperidin or purified hesperetin necessary to treat metabolic syndrome and/or insulin resistance in a subject. A therapeutically effective amount of a purified hesperidin or purified hesperetin or a purified derivative or analog of either compound can be administered in a single dose, or in several doses, for example daily, during a course of treatment. However, the therapeutically effective amount will be dependent on the subject being treated, the severity and type of the affliction, and the manner of administration of the therapeutic(s).

The present disclosure also includes combinations of a purified hesperidin or purified hesperetin or a purified derivative or analog of either compound with one or more other agents useful in the treatment of metabolic syndrome and/or insulin resistance. For example, the compounds of this disclosure can be administered in combination with effective doses of antihypertensives (such as diuretics, beta blockers, angiotensin converting enzyme inhibitors, or calcium channel blockers), lipid lowering compounds (such as statins or fibrates), anti-platelet agents (such as aspirin or P2Y12 receptor antagonists), and/or anti-diabetic agents (such as biguanides, thiazolidinediones, or incretins). The term “administration in combination” or “co-administration” refers to both concurrent and sequential administration of the active agents.

A subject that has metabolic syndrome (for example, as defined by NCEP ATPIII or the International Diabetes Foundation) or a subject with insulin resistance (for example, a fasting plasma glucose of >100 mg/dL) is a candidate for treatment using the therapeutic methods disclosed herein.

The following examples are provided to illustrate certain particular features and/or embodiments. These examples should not be construed to limit the disclosure to the particular features or embodiments described.

### EXAMPLES

Increased consumption of foods rich in flavonoid polyphenols is associated with reduced cardiovascular morbidity and mortality and/or improved cardiovascular health (Hertog et al., Lancet, 342:1007-11, 1993; Hertog et al., Arch. Intern. Med. 155:381-6, 1995; Mink et al., Am. J. Clin. Nutr. 85:895-909, 2007). Biochemical and physiological mechanisms regulating vascular and metabolic actions of the green tea polyphenol EGCG to stimulate production of NO in vascular endothelial cells (Kim et al., J. Biol. Chem. 282:13736-45, 2007), inhibit gluconeogenesis in hepatocytes (Collins et al., J. Biol. Chem. 282:30143-9, 2007), and improve blood pressure, endothelial dysfunction, and insulin resistance in SHR rats (Potenza et al., Am. J. Physiol. Endocrinol. Metab. 292:E1378-87, 2007) were recently elucidated. Therefore, disclosed herein are molecular and cellular mechanisms of action for hesperetin to stimulate production of NO from endothelial cells. A translational clinical study demonstrating that oral hesperidin treatment significantly improved endothelial function, attenuated circulating inflammatory markers and soluble adhesion molecules, lowered total cholesterol and apo-B levels, and increased HDL in subjects with the metabolic syndrome is disclosed herein.

### Example 1

### Hesperetin Stimulates Nitric Oxide Production In Vitro

This example demonstrates that hesperetin stimulates nitric oxide production in cultured endothelial cells and decreases TNF-α-stimulated increases in expression of VCAM-1 and adhesion of monocytes to endothelial cells in culture.

### Materials and Methods

Cell Culture:

Bovine aortic endothelial cells (BAEC) in primary culture (Cell Applications, San Diego, Calif.) were grown in EBM (Endothelial Basal Medium) containing EGM-MV supplements (Endothelial Growth Medium-Micro Vascular; Cambrex, Walkersville, Md.) and used between passages 3 and 5 as previously described (Formoso et al., Mol Endocrinol, 20:1153-63, 2006).

Evaluation of NO Production in Fixed Cells:

Production of NO in BAECs was assessed using the NO-specific fluorescent dye 4,5-diaminofluorescein diacetate (DAF-2 DA, Cayman Chemical, Ann Arbor, Mich.) as described (Kim et al., J. Biol. Chem. 282:13736-45, 2007; Formoso et al., Mol. Endocrinol. 20:1153-63, 2006). Briefly, BAECs were grown to 95% confluence in Lab-Tek™ chamber slides (Rochester, N.Y.) and then serum-starved overnight in phenol red-free EBM supplemented with L-arginine (100 μM). BAECs were loaded with DAF-2 DA (3 μM) for 20 minutes at 37° C. In some experiments, L-N^{G}-Nitroarginine methyl ester (L-NAME; 100 μM), Wortmannin (100 nM), Compound C (5 μM), or PP2 (1 μM) were added to the medium 30 minutes before loading with DAF-2 DA. After loading with DAF-2 DA for 20 minutes, cells were washed with EBM at 37° C. and kept in the dark. Next, BAECs were treated with insulin (100 nM, 5 minutes), lysophosphatidic acid (LPA; 5 μM, 5 minutes), or hesperetin (10 μM, 10 minutes; Sigma). After treatment, cells were fixed in 4% paraformaldehyde (vol/vol) for 5 minutes at 4° C. Fixed cells were visualized using an Olympus IX81 inverted microscope with attached CCD camera (Retiga Exi, Burnaby, BC, Canada) using appropriate filters with a peak excitation wavelength of 480 nm and a peak emission wavelength of 510 nm. Images were captured using IPLab™ Software (Scanalytics, Inc., Fairfax, Va.).

Immunoblotting:

BAECs were grown in 60 mm dishes, serum-starved overnight, and then treated with hesperetin (10 μM) for varying durations or for 10 minutes at different concentrations. In some experiments, Wortmannin (100 nM), or Compound C (5 μM) was added to cells 1 hour before treating with hesperetin. Cell lysates were prepared using 120 μl of lysis buffer (100 mM NaCl, 20 mM Hepes, pH 7.9, 1% Triton X-100, 1 mM Na₃VO₄, 4 mM sodium pyrophosphate, 10 mM EDTA, 1 mM phenylmethylsulfonyl fluoride, 10 mM NaF, and complete protease inhibitor cocktail (Roche Applied Sciences, Indianapolis, Ind.)). After centrifugation of samples at 17,000×g for 5 minutes at 4° C., supernatants were boiled with Laemmli sample buffer for 5 minutes and proteins (50 μg total protein) were resolved by 10% SDS-PAGE, transferred to nitrocellulose membranes, and immunoblotted using antibodies against eNOS (Transduction Laboratories), phospho-eNOS (Ser¹¹⁷⁹), Akt, phospho-Akt (Ser⁴⁷³), AMPK, phospho-AMPK (Thr¹⁷²), Src (Tyr⁴¹⁸) (Cell Signaling Technology), VCAM-1 (Santa Cruz, Calif.) or β-actin (Sigma) according to standard methods. Immunoblots were quantified by scanning densitometry (GE Healthcare).

Monocyte Adhesion Assay:

BAECs were cultured in 6-well plates and then treated with TNF-α (10 ng/ml, 5 hours) or hesperetin (10 μM, 5 hours). In some experiments, hesperetin was added 1 hour before treating cells with TNF-α. U937 monocytes were grown in DMEM medium containing 10% FBS in a culture flask. U937 monocytes were labeled for 15 min with 5 μM calcein-AM (Molecular Probes, Inc.). Labeled U937 cells (6×10⁵ cells) were then incubated for 30 minutes at 37° C. with confluent BAECs pre-treated with hesperetin and/or TNF-α. Co-cultured cells were washed three times with PBS and then images of monocytes adhering to BAECs were obtained with an Olympus IX81 inverted microscope with attached CCD camera (Retiga Exi, Burnaby, BC, Canada) using appropriate filters. Images were captured using IPLab™ Software (Scanalytics, Inc., Fairfax, Va.).

Measurement of H₂O₂/ROS Production:

Production of intracellular H₂O₂ was assessed using the H₂O₂-specific fluorescent dye 5-(and -6)-chloromethyl-2,7-dichlorodihydrofluorescein diacetate (CM-H₂DCF-DA, Invitrogen) as previously described (Kim et al., J Biol Chem, 282:13736-45, 2007). Briefly, BAECs were plated on chamber slides and incubated overnight in media free of phenol red, serum, and growth factors. Cells were then washed with Dulbecco's phosphate buffered saline containing calcium and magnesium (DPBS) before each experiment. Cells were loaded with CM-H₂DCF-DA (5 μM, 20 minutes, 37° C.) and then washed to remove excess CM-H₂DCF-DA. Cells were then treated with H₂O₂, hesperetin, or naringenin as indicated in the legends to figures followed by washing three times with DPBS. Some groups of cells were pre-treated with N-acetylcysteine (NAC) (10 mM, 2 hours). Cells were then visualized and images were captured as described for measurement of NO production.

### Results

Hesperetin Acutely Stimulates Phosphorylation of Akt, AMPK, and eNOS to Mediate NO Production in Vascular Endothelial Cells:

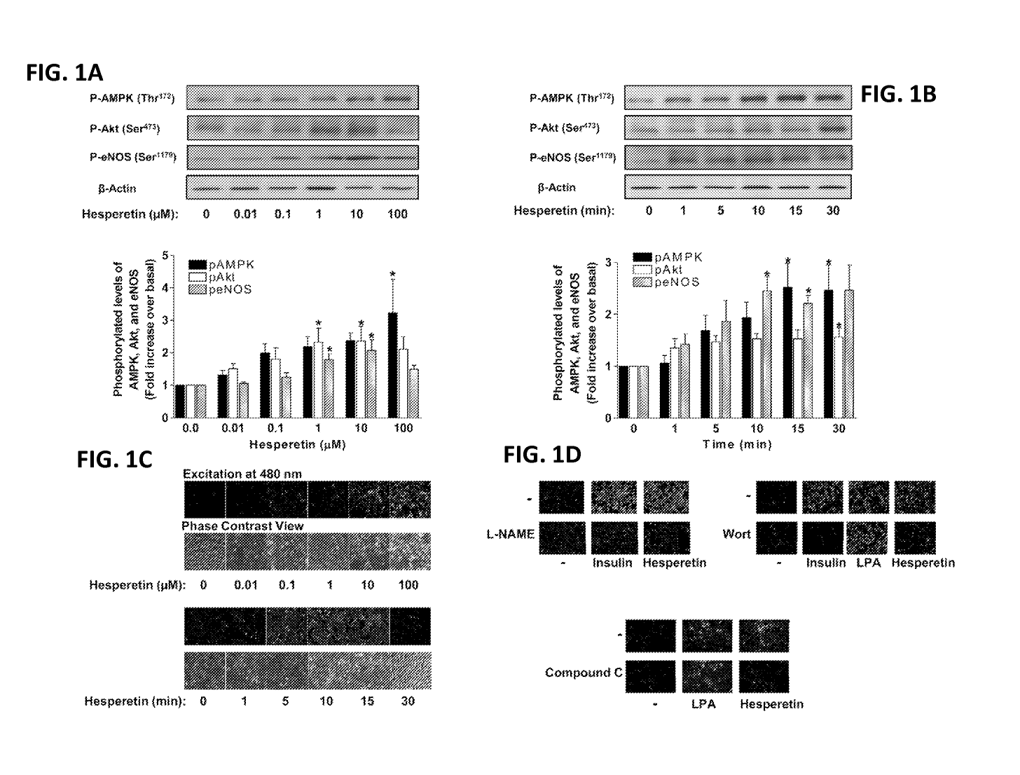

Treatment of bovine aortic endothelial cells (BAEC) in primary culture with hesperetin acutely increased cellular levels of phosphorylated AMPK (Thr¹⁷²) and Akt (Ser⁴⁷³) (indicative of enzyme activation) in a concentration- and time-dependent fashion (FIGS. 1A and B). Both AMPK and Akt regulate activity of eNOS by phosphorylating Ser¹¹⁷⁹ resulting in increased production of NO (Dimmeler et al., Nature, 399:601-5, 1999, Chen et al., FEBS Lett, 443:285-9, 1999). Accordingly, hesperetin treatment of BAEC also increased phospho-eNOS (Ser¹¹⁷⁹) with a corresponding concentration- and time-dependent increase in hesperetin-stimulated NO production in BAEC (FIG. 1C). Peak production of NO paralleled eNOS phosphorylation and occurred after 5-10 minutes of hesperetin treatment. Pre-treatment of cells with the NO synthase inhibitor L-NAME blocked NO production in response to insulin (positive control) or hesperetin (FIG. 1D).

Insulin phosphorylates and activates eNOS through a PI3K/Akt-dependent pathway (Zeng et al., Circulation, 101:1539-45, 2000). By contrast, LPA (a phospholipid growth factor that mobilizes intracellular calcium) activates eNOS without phosphorylating eNOS (Montagnani et al., J Biol Chem, 276:30392-8, 2001). Importantly, pre-treatment of BAEC with wortmannin (an inhibitor of PI3K) inhibited insulin- (positive control) and hesperetin- but not LPA- (negative control) stimulated production of NO. Compound C, an inhibitor of AMPK, substantially inhibited production of NO in response to hesperetin but not to LPA (negative control) (FIG. 1D). Thus, similar to the green tea polyphenol EGCG, the citrus polyphenol hesperetin stimulated PI3K that resulted in activation of downstream serine kinases Akt and AMPK to phosphorylate and activate eNOS to produce NO in vascular endothelium (Kim et al., J Biol Chem, 282:13736-45, 2007).

Additional Signalling Mechanisms Determining Hesperetin-stimulated Production of NO:

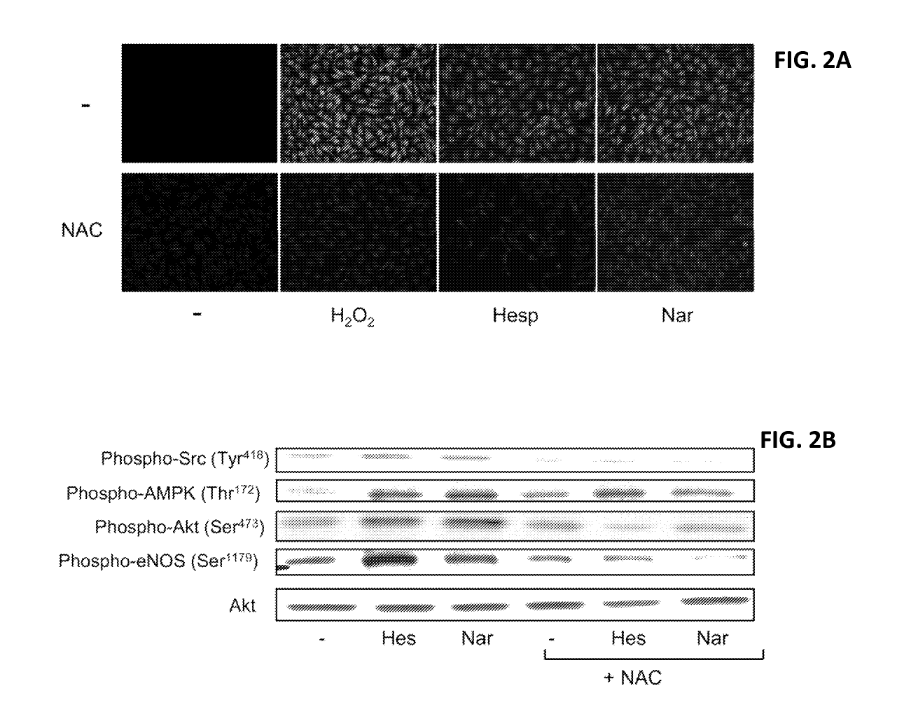

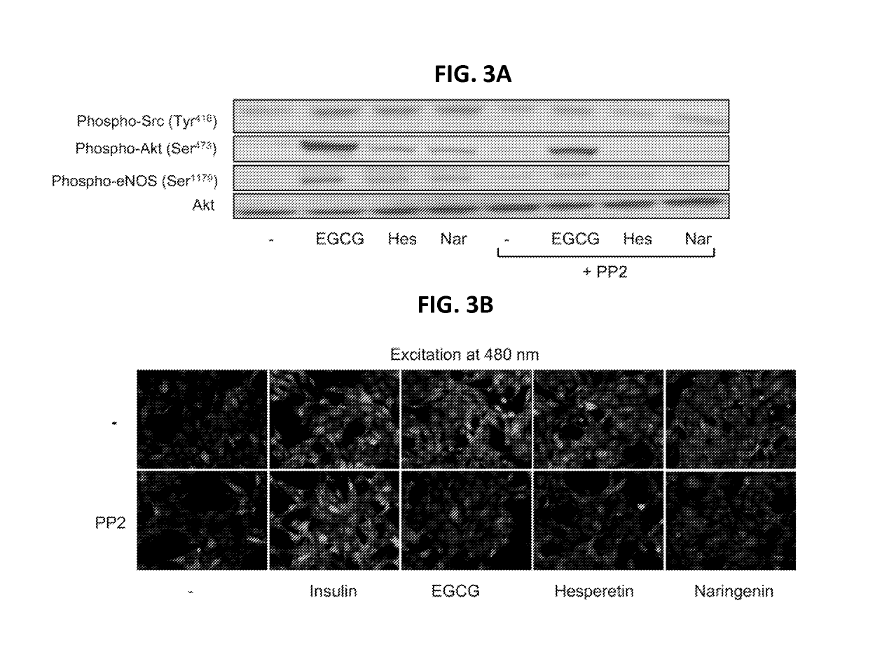

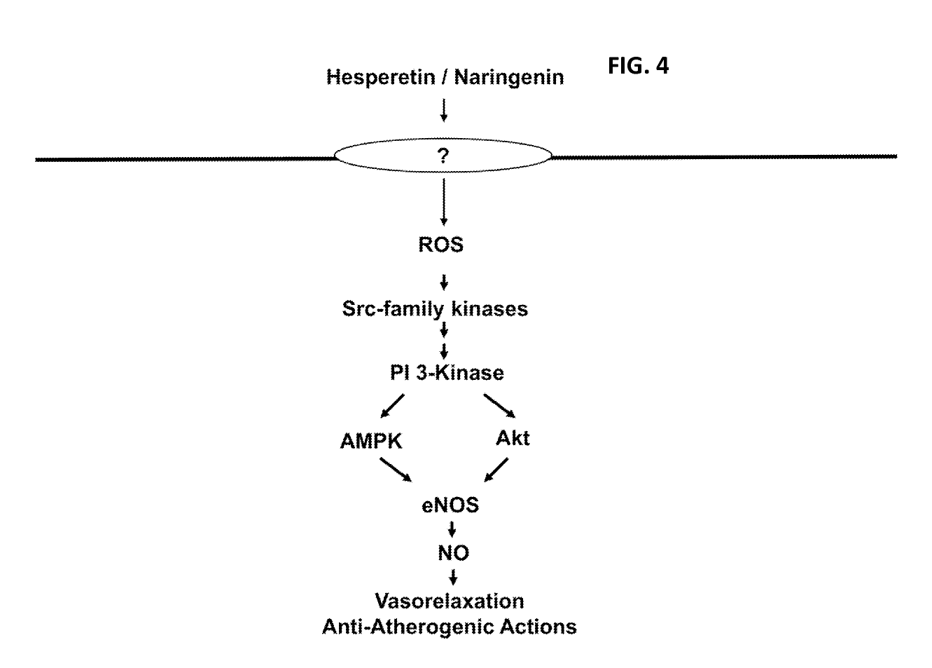

It has previously been demonstrated that EGCG stimulates production of ROS leading to activation of Fyn upstream of PI3K/Akt/eNOS in endothelial cells (Kim et al., J Biol Chem, 282:13736-45, 2007). Hesperetin (and the related citrus polyphenol, naringenin) also stimulated production of ROS in BAEC (FIG. 2A). Moreover, pre-treatment of BAEC with N-acetylcysteine (NAC) scavenged the production of ROS and inhibited hesperetin and naringenin-stimulated phosphorylation of Src (Tyr⁴¹⁸), Akt (Ser⁴⁷³), and eNOS (Ser¹¹⁷⁹) (FIG. 2B). Similar to EGCG (positive control), hesperetin-stimulated phosphorylation of Src, Akt, and eNOS, as well as production of NO, were all inhibited by pre-treatment of BAEC with the Src inhibitor PP2 (FIGS. 3A and B), further supporting the role of Src-family kinases. By contrast, PP2 pre-treatment failed to inhibit insulin-stimulated NO production (FIG. 3B; negative control; insulin activates PI3K/Akt/eNOS in a src-independent fashion; Kim et al., J Biol Chem, 282:13736-45, 2007). Thus, signaling mechanisms by which hesperetin/naringenin activated eNOS to regulate production of NO in endothelial cells were similar to those previously reported for EGCG (Kim et al., supra) (FIG. 4).

Effects of Hesperetin on Expression of Adhesion Molecules and Monocyte Adhesion to Endothelium:

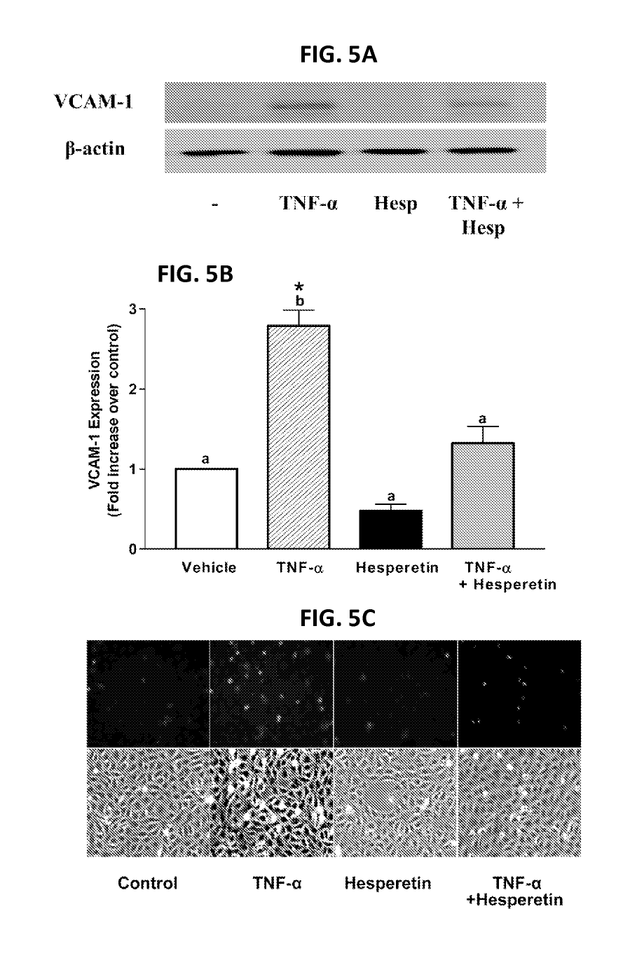

Endothelial production of NO exerts anti-atherogenic, anti-thrombotic, and anti-inflammatory actions (Napoli et al., Nitric Oxide, 15:265-79, 2006). Therefore, the potential of hesperetin pre-treatment to reduce TNF-α-stimulated expression of VCAM-1 and adhesion of monocytes to endothelial cells was examined (FIG. 5). When compared with vehicle-treated control cells, treatment of BAEC with the pro-inflammatory cytokine TNF-α increased expression of VCAM-1 with a corresponding increase in adhesion of labeled monocytes. Importantly, hesperetin pre-treatment of BAEC substantially reduced these pro-atherogenic actions of TNF-α. Thus, in addition to stimulating acute production of NO from endothelium, hesperetin has anti-atherogenic actions that may help attenuate endothelial dysfunction and oppose atherogenic actions of pro-inflammatory cytokines.

### Example 2

### Effect of Oral Hesperidin on Metabolic Syndrome Parameters

This example demonstrates the effect of oral hesperidin treatment on endothelial function, circulating inflammatory markers and soluble adhesion molecules, and metabolic parameters in subjects with metabolic syndrome.

### Materials and Methods

Study Design and Study Subjects:

A randomized, placebo-controlled, double-blind, crossover trial of hesperidin (500 mg p.o. daily for 3 weeks) in individuals with metabolic syndrome was conducted. This study was conducted exclusively at the Clinical Center for Atherosclerosis at the University of Rome “Tor Vergata,” Rome, Italy (ClinicalTrials.gov Identifier: NCT00914251). The study protocol was approved by the Institutional Ethics Board, and all procedures followed were in accordance with institutional guidelines. Adults between 21 and 65 years of age, with metabolic syndrome (National Cholesterol Education Program Adult Treatment Panel [NCEP ATP] III criteria) were recruited from the local community through newspaper advertisements.

Subjects were specifically excluded from study enrolment if they required initiation of pharmacologic treatment for diabetes, hypertension, or dyslipidemia within two months before randomization, if they were pregnant or had liver disease, pulmonary disease, renal insufficiency, coronary heart disease, heart failure, peripheral vascular disease, coagulopathy, any other severe systemic diseases, or if they were allergic to citrus fruits. Subjects were also excluded if they were under treatment for or had a history of any form of cancer, or if they had positive blood tests for HIV, hepatitis B, or hepatitis C. All of the participating women reported regular menstrual cycles without oral contraceptive therapy. In female subjects, all experiments were performed during the first week of the menstrual cycle.

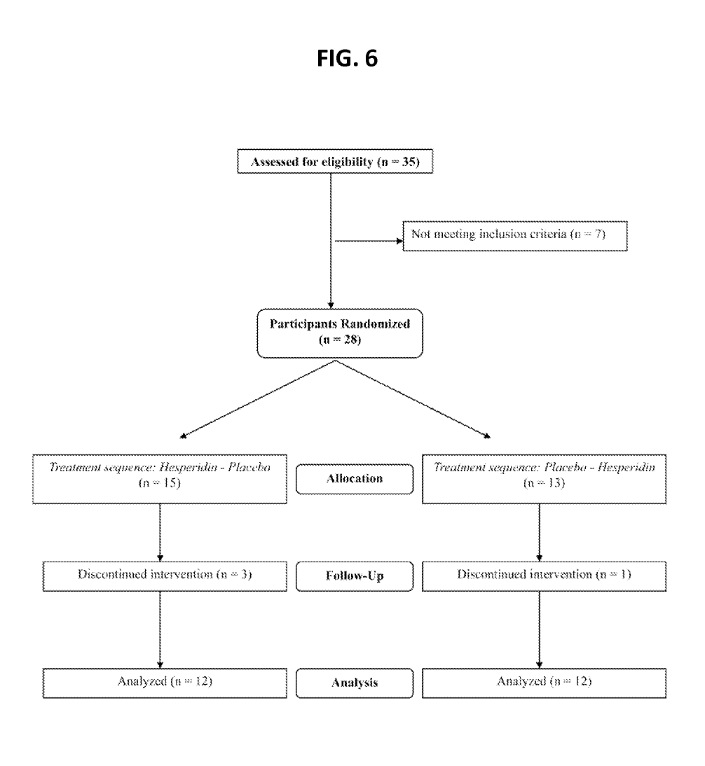

Of the 35 individuals screened, 28 were deemed eligible for the study (FIG. 6). Informed consent was obtained from each subject. Enrolled subjects were randomly assigned in a double-blind fashion (block randomization by Clinical Center Pharmacy) to the initial arm of the study consisting of either hesperidin (500 mg/d) or matching placebo for three weeks. This was followed by a three-day washout period. Subjects were then crossed over to the other treatment arm for an additional three weeks. Endothelial function, metabolic parameters, and markers of inflammation were assessed at baseline and after each three-week treatment period. Compliance with treatment was assessed by capsule counts at the end of the study. Study investigators and participants were blinded to treatment assignment and assignment codes were not available to investigators until the entire study and the database had been completed and secured. Participants were instructed to maintain their usual physical activity and dietary habits. No additional medications (apart from medications at baseline) were allowed during the study period to avoid confounding effects of other drugs.

Hesperidin/Placebo Description:

Hesperidin (98% pure, Citrus sinensis) was from Blue California (Rancho Santa Margarita, Calif.). Capsules containing 500 mg hesperidin and matching placebo capsules containing cellulose were formulated by the pharmacy at Clinical Center for Atherosclerosis at the University of Rome “Tor Vergata,” Rome, Italy. The dose (500 mg once a day) and duration (three weeks) of hesperidin treatment was based on available safety data in humans (Miwa et al., J Nutr Sci Vitaminol (Tokyo), 50:211-8, 2004; Miwa et al., J Nutr Sci Vitaminol (Tokyo), 51:460-70, 2005).

Endothelial Function Testing using Flow-mediated Dilation:

Assessment of endothelial function was conducted in the fasting state (24 hours after the last dose of hesperidin or placebo) using a standardized procedure at approximately the same point in time and before all of the scheduled metabolic tests. All studies were performed in the morning in a quiet room with a temperature of about 22° C. Participants were asked to refrain from drinking alcohol or beverages containing caffeine for at least 24 hours before the test, and all study subjects fasted for at least 10 hours before the study day. Endothelium-dependent and -independent vasodilator functions were assessed as previously reported (Rizza et al., Atherosclerosis, 206:569-74, 2009; Tesauro et al., Metabolism, 56:413-9, 2007). Briefly, subjects lay supine on a bed and were allowed to rest for at least 10 minutes. Then, the left brachial artery was visualized 2-15 cm proximal to the antecubital fossa with high-resolution ultrasound (ATL HDI 3000, with a 7.5-MHz linear array transducer, Philips Medical Systems, Best, Netherlands). After baseline images and flow measurements were obtained, a pressure cuff applied on the upper arm was inflated to 200-250 mm Hg for 5 minutes. Blood flow was measured during the 15 seconds following cuff release, and arterial images that measured diameter were acquired between 60 and 90 seconds after cuff deflation. Flow-mediated dilation (FMD) was calculated as the increase in post-stimulus diameter as a percentage of the baseline diameter. After at least a 15-minute rest, endothelium-independent vasomotor responsiveness was assessed by acquiring images and flow measurements before and after administration of 0.4 mg sublingual nitroglycerin. Blood flow and images for arterial diameter were recorded between 3 and 4 minutes after nitroglycerin administration. The nitroglycerin-mediated dilation (NMD) was calculated as the increase in post-stimulus diameter as a percentage of the baseline diameter. For both FMD and NMD, arterial diameter was measured from the anterior to the posterior endothelial-lumen interface at the end diastole, coincident with the R wave on the electrocardiogram. Images were then coded and analyzed by an investigator blinded to the image sequence and to the treatment arm of subjects.

Circulating Endothelial Adhesion Molecules and Pro-inflammatory Markers:

Blood samples were obtained from all patients in the fasting state and serum was stored at −80° C. until analysis. Samples were divided in aliquots that were frozen and thawed only once. The following circulating markers were evaluated: serum high sensitivity C reactive protein (hs-CRP; nephelometric assay, Dade-Behring, Liederbach, Germany); Serum Amyloid A Protein (SAA, Dade-Behring, U.S.A); and homocysteine (ADVIA centaur). Circulating concentrations of sVCAM-1, sICAM-1, and sE-selectin (ELISA; Bender MedSystem) were measured by ELISA using high sensitivity commercial kits. All assays were carried out on the same day to minimize assay variability.

Laboratory Assays:

Routine assays for serum lipids, plasma glucose and insulin, and HbA_{1C} were performed in the Clinical Center for Atherosclerosis at the University of Rome “Tor Vergata,” Rome, Italy.

Statistical Analyses:

Data from participants who completed all phases of the protocol (n=24) were analyzed according to a pre-established statistical analysis plan. The primary outcome measure for this study was prospectively designated as the difference in FMD between placebo and hesperidin treatment periods. All other comparisons were considered secondary. Therefore, power analysis was calculated for this outcome in this 2-treatment crossover study. A sample size of n=20 was deemed sufficient to provide 80% power in detecting a difference of 2.0% or greater in FMD of the brachial artery between placebo and hesperidin, with α=0.05 based on previous studies (Rizza et al., Atherosclerosis, 206:569-74, 2009). The presence of skewed data was evaluated by visual inspection of Q-Q plots, stem and leaf plots, or box plots, and verified by the Shapiro-Wilk test for normal distribution. After subtracting baseline observations, comparison of various post-treatment parameters was performed using crossover ANOVA (Armitage P, Berry G, Matthews J. Statistical Methods in Medical Research. 4th edition ed., Oxford: Blackwell Science, 1994). This analysis takes into account specific treatment arm, treatment order, and treatment effects. Thus, carryover effects in the crossover study were explicitly tested for. Because of the exploratory nature of this clinical study, no adjustments were made for multiple comparisons. The statistical software StatsDirect version 2.7.2 (Cheshire, UK) was used for data analysis.

### Results

Translational Clinical Study with Hesperidin:

Based upon novel cellular mechanisms of action of hesperetin in endothelial cells (Example 1), an exploratory translational clinical intervention study was designed to evaluate whether oral hesperidin administration improves endothelial dysfunction and circulating markers of inflammation in subjects with metabolic syndrome. From among 35 individuals screened, 28 were deemed eligible for study run-in. Among 28 subjects randomized, 24 subjects completed the entire study. One subject was lost to follow-up and three subjects withdrew after recommendation by their personal physicians (FIG. 6). There were no adverse effects reported by the study participants either during the placebo or hesperidin treatment periods.

Baseline Clinical Characteristics of Study Subjects:

This study used a sequential cross-over design where each study participant received either placebo or hesperidin first in random order, followed by a wash-out period, and then crossover to the other treatment arm. Baseline clinical characteristics of 24 subjects who completed our entire study are reported in Table 1. Baseline medications included oral anti-diabetic therapy (71%, n=17), anti-hypertensive therapy (42%, n=10), statin therapy (4%, n=1), and anti-platelet therapy (13%, n=3) and were used in similar proportions in subjects assigned to each initial treatment arm. As expected for patients with metabolic syndrome, subjects were obese, hypertensive, and insulin resistant.

TABLE 1
Baseline clinical characteristics of study subjects

| | Overall | | Placebo- | | Hesperidin- | |
|---|---|---|---|---|---|---|
| Clinical | cohort | | Hesperidin | | Placebo | |
| Parameters | (n = 24) | | (n = 12) | | (n = 12) | |
| Age (years) | 52 ± 2 | | 50 ± 3 | | 53 ± 1 | |
| Gender (M/F) | 15/9 | | 6/6 | | 9/3 | |
| Waist | 112 ± 3 | | 114 ± 4 | | 110 ± 4 | |
| circumference (cm) | | | | | | |
| Body Mass | 34.7 ± 1.5 | | 35.4 ± 2.0 | | 33.9 ± 2.2 | |
| Index (kg/m²) | | | | | | |
| Vascular Parameters | | | | | | |
| Systolic BP | 138 ± 3 | | 136 ± 4 | | 139 ± 5 | |
| (mm Hg) | | | | | | |
| Diastolic BP | 89 ± 2 | | 90 ± 3 | | 88 ± 3 | |
| (mm Hg) | | | | | | |
| FMD (%) | 8.24 ± 0.88 | | 8.61 ± 1.15 | | 7.87 ± 1.40 | |
| NMD (%) | 13.98 ± 1.32 | | 15.75 ± 1.93 | | 12.21 ± 1.73 | |
| Metabolic parameters | | | | | | |
| FPG (mg/dL) | 132 ± 12 | | 116 ± 10 | | 148 ± 21 | |
| FPI (μU/dL) | 21.3 ± 2.1 | | 23.2 ± 2.8 | | 19.3 ± 2.9 | |
| QUICKI | 0.298 ± 0.005 | | 0.296 ± 0.005 | | 0.299 ± 0.008 | |
| Hemoglobin | 6.6 ± 0.2 | | 6.1 ± 0.3 | | 7.1 ± 0.3 | |
| A1C (%) | | | | | | |
| Lipids (mg/dL) | | | | | | |
| Total cholesterol | 179 ± 8 | | 194 ± 12 | | 165 ± 11 | |
| Low density | 119 ± 8 | | 131 ± 10 | | 107 ± 11 | |
| lipoprotein | | | | | | |
| Apolipoprotein B | 90 ± 4 | | 92 ± 6 | | 89 ± 7 | |
| Lipoprotein(a) | 9.4 | (2.5-21.7) | 9.0 | (3.7-19.9) | 11.6 | (6.8-32.2) |

| | | | |
|---|---|---|---|
| High density | 37 ± 2 | 42 ± 2 | 33 ± 3 |
| lipoprotein | | | |
| Apolipoprotein A-I | 134 ± 5 | 148 ± 5 | 121 ± 7 |
| Triglycerides | 158 ± 11 | 160 ± 17 | 157 ± 14 |

Values are mean ± SEM or median (25^{th}-75^{th} percentile); n, number of subjects; BP, blood pressure; FMD, flow-mediated dilation; NMD, nitroglycerin-mediated dilation; FPG, fasting plasma glucose; FPI, fasting plasma insulin; QUICKI, quantitative insulin-sensitivity check index.

Effects of Hesperidin Treatment on Vascular Parameters:

When compared with placebo treatment, three-week treatment with hesperidin caused significant improvement in FMD (Table 2). Vasodilation of the brachial artery in response to sublingual nitroglycerin was not significantly different between placebo and hesperidin treatment (Table 2). When compared with baseline values, FMD values at the end of the placebo-treatment period were not significantly different (Table 2; p=0.63), indicating no substantial deterioration of endothelial function. Moreover, a posteriori comparison of FMD values at baseline and at the end of the hesperidin-treatment period suggest that administration of hesperidin significantly improved endothelial function in our cohort (Table 2; p=0.05). Thus, using an additional analysis that does not consider placebo treatment, hesperidin treatment also improved endothelial function (over baseline). Using appropriate statistical analyses, no significant carry-over effects were observed (i.e., treatment-period interactions were non-significant) between initial hesperidin or placebo treatments arms with respect to any of the measured vascular parameters in the cross-over study.

TABLE 2
Effect of hesperidin treatment on vascular function in study subjects

| Vascular Parameters | | | | | | |
|---|---|---|---|---|---|---|
| (n = 24) | Baseline | Placebo | Hesperidin | Treatment Effect | | P value |
| Systolic BP (mm Hg) | 138 ± 3 | 132 ± 2 | 134 ± 3 | 2.7 | (−1.256.66) | 0.16 |
| Diastolic BP (mm Hg) | 89 ± 2 | 90 ± 2 | 90 ± 2 | 0.6 | (−2.1-3.4) | 0.64 |
| FMD (%) | 8.24 ± 0.88 | 7.78 ± 0.76 | 10.26 ± 1.19 | 2.47 | (0.39-4.55) | 0.02 |
| NMD (%) | 13.98 ± 1.32 | 14.4 ± 1.02 | 14.04 ± 1.08 | −0.35 | (−1.03-0.32) | 0.30 |

Values shown at baseline and after treatment with placebo or hesperidin are mean ± SEM or median (25^{th}-75^{th} percentile); n, number of subjects. After subtracting baseline observations, comparison of various post-treatment parameters was performed using crossover ANOVA. This analysis takes into account specific treatment arm, treatment order, and treatment effects. Treatment effects are expressed as mean (95% CI) or ratio (95% CI). P values are for post-treatment comparisons (placebo vs. hesperidin). FMD, flow-mediated dilation; NMD, nitroglycerin-mediated dilation.

Effects of Hesperidin Treatment on Circulating Inflammatory Markers and Soluble Adhesion Molecules:

Based upon the cellular studies (Example 1), mechanisms whereby hesperidin treatment may improve endothelial dysfunction were examined by measuring circulating levels of inflammatory markers and soluble adhesion molecules in the study subjects (Table 3). When compared with placebo treatment, three week treatment with hesperidin significantly reduced circulating concentrations of hs-CRP, SAA protein, and sE-Selectin. Post-placebo treatment levels of vascular adhesion molecules were not significantly different from baseline levels (p>0.33). Similarly, post-placebo treatment levels of hsCRP and SAA protein were not significantly different from baseline levels (hsCRP, p=0.24, SAA, p=0.32). Direct comparisons of post-hesperidin treatment values with baseline levels indicate that hesperidin treatment reduces circulating levels of hsCRP, SAA, and E-selectin (hsCRP, p=0.007; SAA, p=0.002; sE-Selectin, p=0.002).

TABLE 3
Effect of hesperidin treatment on circulating inflammatory markers and vascular adhesion molecules

| Circulating | | | | | | | Treatment | | P |
|---|---|---|---|---|---|---|---|---|---|
| Biomarkers(n = 24) | Baseline | | Placebo | | Hesperidin | | Effect | | value |
| Inflammatory markers | | | | | | | | | |
| hsCRP (mg/L) | 3.9 | (2.0-6.5) | 4.9 | (2.3-7.4) | 2.6 | (1-5.7) | 0.68 | (0.51-0.91) | 0.01 |
| SAA (mg/L) | 7.3 | (5.6-6.1) | 8.0 | (5.6-11.2) | 5.6 | (3.2-7.8) | 0.62 | (0.47-0.82) | 0.001 |
| Fibrinogen (mg/dL) | 320 ± 14 | | 330 ± 16 | | 331 ± 15 | | 0.6 | (−19.9-21.2) | 0.94 |
| Homocysteine (μM/L) | 11.9 | (10.3 − 14.9) | 13.6 | (10.6-16.7) | 13.0 | (10.2-15.5) | 0.95 | (0.87-1.05) | 0.30 |

| Vascular adhesion | | | | | | |
|---|---|---|---|---|---|---|
| molecules (ng/mL) | | | | | | |
| VCAM | 956 ± 29 | 976 ± 30 | 950 ± 27 | −28 | (−71-16) | 0.19 |
| ICAM | 291 ± 6 | 299 ± 7 | 294 ± 7 | −4 | (−19-11) | 0.59 |
| Soluble E-selectin | 31 ± 2 | 31 ± 2 | 27 ± 2 | −4 | (−6-−1) | 0.002 |

Values shown at baseline and after treatment with placebo or hesperidin are mean ± SEM or median (25^{th} -75^{th} percentile); n, number of subjects. Statistical analyses were performed as described in Table 2. Treatment effects are expressed as mean (95% CI) or ratio (95% CI). P values are for post-treatment comparisons (placebo vs. hesperidin). SAA, serum amyloid A protein; VCAM, vascular cell adhesion molecule; ICAM, intracellular adhesion molecule.

Effects of Hesperidin Treatment on Metabolic Parameters:

When compared with placebo treatment, three week treatment with hesperidin caused significant decreases in circulating concentrations of total cholesterol and apolipoprotein B (apo B), and increased HDL (Table 4). No significant differences were observed in LDL, lipoprotein [a], apolipoprotein A-I (apoA-I), triglycerides, fasting plasma glucose, or fasting plasma insulin concentrations. Hesperidin treatment caused a trend towards improving insulin resistance as assessed by QUICKI (p=0.06).

TABLE 4
Effect of hesperidin treatment on metabolic parameters in study subjects.

| Metabolic | | | | | | | | | |
|---|---|---|---|---|---|---|---|---|---|
| Parameters | | | | | | | Treatment | | P |
| (n = 24) | Baseline | | Placebo | | Hesperidin | | Effect | | value |
| BMI (kg/m²) | 34.7 ± 1.5 | | 34.7 ± 1.5 | | 34.7 ± 1.5 | | 0.004 | (−0.2-0.2) | 0.95 |
| Waist circumf. | 112 ± 3 | | 112 ± 3 | | 112 ± 3 | | 0.42 | (−0.15-0.98) | 0.14 |
| (cm) | | | | | | | | | |
| FPG (mg/dL) | 132 ± 12 | | 129 ± 7 | | 126 ± 6 | | −3.66 | (−9.39-2.05) | 0.19 |
| FPI (μU/mL) | 21.3 ± 2.1 | | 21.1 ± 1.9 | | 20.2 ± 2.1 | | −0.86 | (−2.29-0.56) | 0.22 |
| QUICKI | 0.298 ± 0.004 | | 0.297 ± 0.003 | | 0.300 ± 0.004 | | 0.003 | (0.0002-0.006) | 0.06 |
| Hemoglobin | 6.61 ± 0.24 | | 6.63 ± 0.24 | | 6.59 ± 0.24 | | −0.04 | (−0.09-0.01) | 0.12 |
| A1C (%) | | | | | | | | | |
| Lipids (mg/dL) | | | | | | | | | |
| Total cholest. | 179 ± 8 | | 185 ± 9 | | 174 ± 8 | | −11.4 | (−21.6-−1.1) | 0.03 |
| LDL | 119 ± 8 | | 122 ± 6 | | 115 ± 6 | | −7 | (−16-2) | 0.11 |
| ApoB | 90 ± 4 | | 93 ± 4 | | 88 ± 4 | | −4.79 | (−9.36-−0.22) | 0.04 |
| Lipoprotein(a) | 9.4 | (2.5-21.7) | 12.0 | (5.0-24.7) | 10.5 | (5.0-26.5) | 0.87 | (0.70-1.08) | 0.20 |

| | | | | | | |
|---|---|---|---|---|---|---|
| HDL | 37 ± 2 | 34 ± 2 | 35 ± 2 | 1.3 | (−0.01-2.51) | 0.05 |
| ApoA-I | 134 ± 5 | 136 ± 7 | 137 ± 6 | 0.33 | (−4.94-5.61) | 0.89 |
| TG | 158 ± 11 | 179 ± 17 | 164 ± 10 | −15 | (−44-14) | 0.28 |

Values shown at baseline and after treatment with placebo or hesperidin are mean ± SEM or median (25^{th}-75^{th} percentile); n, number of subjects. Statistical analyses were performed as described in Table 2. Treatment effects are expressed as mean (95% CI) or ratio (95% CI). P values are for post-treatment comparisons (placebo vs. hesperidin). LDL, low density lipoprotein; ApoB, apolipoprotein B; HDL, high density lipoprotein; ApoA-I, apolipoprotein A-I.

### Example 3

### Analysis of Hesperetin in Plasma

This example demonstrates analysis of hesperetin in serum samples by high performance liquid chromatography (HPLC).

### Materials and Methods

Standard Solutions:

Stock solutions of hesperidin, hesperetin and 7-ethoxy-coumarine (Internal Standard) were prepared by dissolving an appropriate amount of each standard with methanol to achieve concentration of 1 mg/ml. Appropriate dilutions of the stock solution of hesperidin and hesperetin were made with methanol/water (1:1 v/v) to prepare the working solutions containing 1.25, 3.75, 5, 10, 12.5, and 15 μg/ml of hesperidin and hesperetin. The internal standard stock solution was diluted with methanol/MilliQ-water (1:1 v/v) to obtain the working solution at 25 μg/ml.

Calibration Standard Samples:

Calibration standard samples were freshly prepared in 1 ml of human plasma spiked with 20 μl of hesperetin working solutions and 20 μL of the internal standard working solution. The final concentrations of hesperetin were: 25, 75, 100, 200, 250 and 300 ng/ml of plasma.

Sample Preparation:

Plasma samples (1 ml) from normal or obese subjects were spiked with hesperetin and internal standard and were incubated with 100 μl of 1 M sodium acetate buffer (pH 5) and 40 μl of β-glucuronidase/sulphatase (crude preparation from H. pomatia, Sigma-Aldrich, St. Louis, Mo.) for 3 hours at 37° C. The hydrolyzed plasma samples were diluted with 2 ml of phosphate buffer (0.1 M, pH 2.4) and then applied to the extraction C18 cartridges (3 ml, 500 mg). Solid phase extraction was conducted as previously reported (Kanaze et al., J. Chromatogr. B 801:363-367, 2004). Briefly the cartridges were preconditioned with 6 ml of methanol and then with 6 ml of 0.01 M HCl; then washed with 5.0 ml of 10% methanol in 0.01 M HCl and then with 3.0 ml of 0.01 M HCl and purged with air. Hesperetin and internal standard were eluted with 1.5 ml (3×0.5 ml) of acetonitrile. The eluate was evaporated to dryness with a gentle nitrogen flow. The residue was dissolved in 200 μl of methanol/MilliQ-water 1:1 v/v and 25 μl was injected into the chromatographic system.

Chromatographic Conditions:

The analyses were performed using an HPLC system (Dionex, model P580), an autosampler with a 25 μl loop (Dionex, ASI-100), and a variable wavelength UV-Vis detector (Dionex Ultimate 3000) set at 288 nm. Separation was performed on a Dionex Acclaim® 120 C18 5 μm 120 Å 4.6×150 mm. The mobile phase consisted of A): H₂O/Acetonitrile (95/5)/TFA 0.1% and B) Acetonitrile/TFA 0.1%. The gradient elution program was: 0-15 minutes: 0-50% B; 15-17 minutes: 50-98% B; 17-20 minutes: 98% B; 20-21 minutes: 98%-50% B; 21-25 minutes: 0% B. The flow rate was 1 ml/min.

### Results

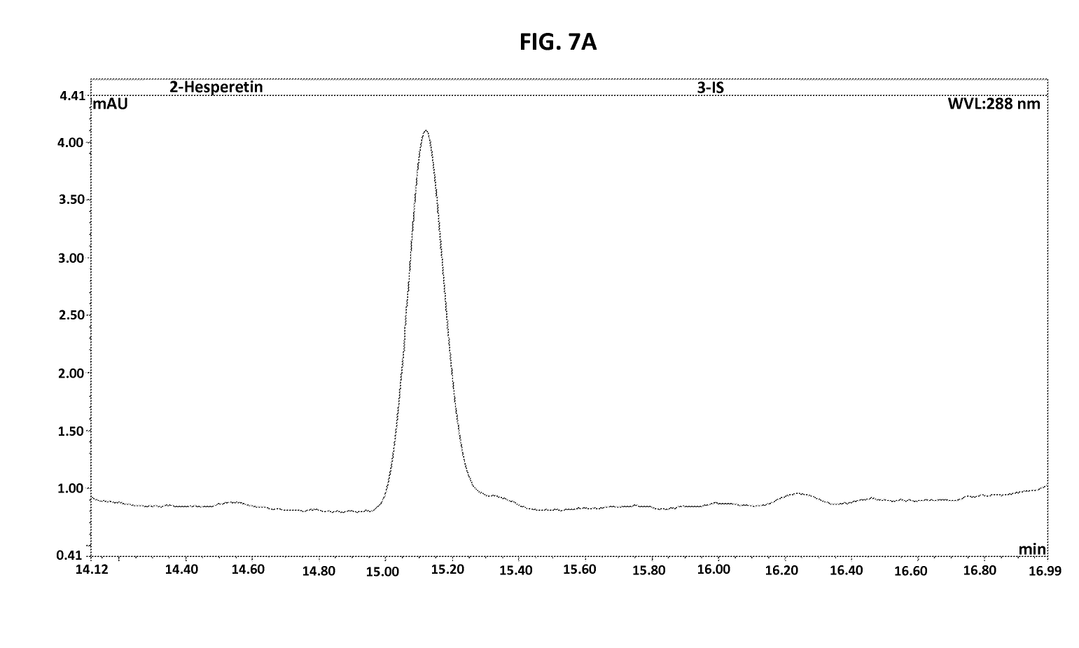

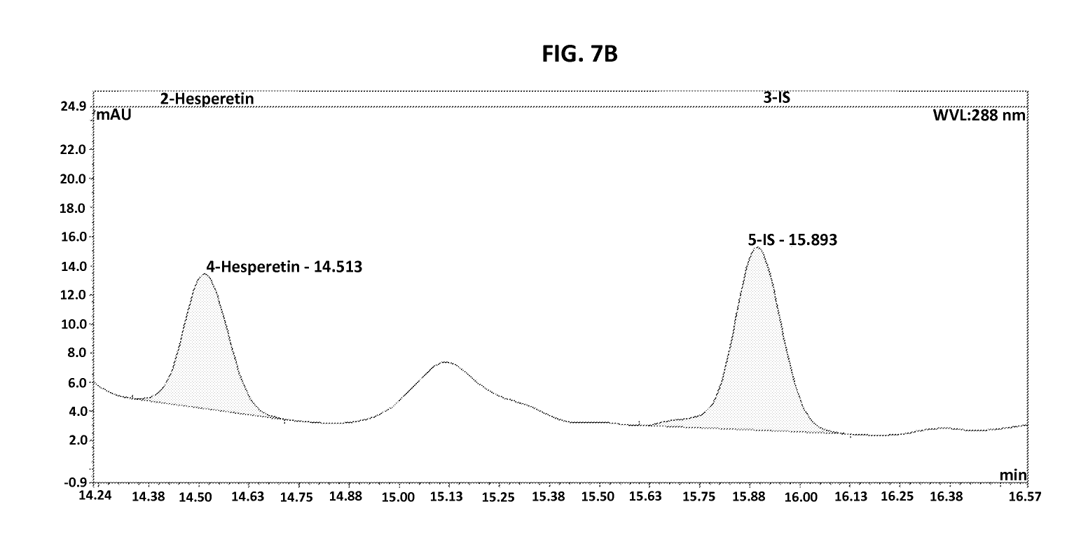

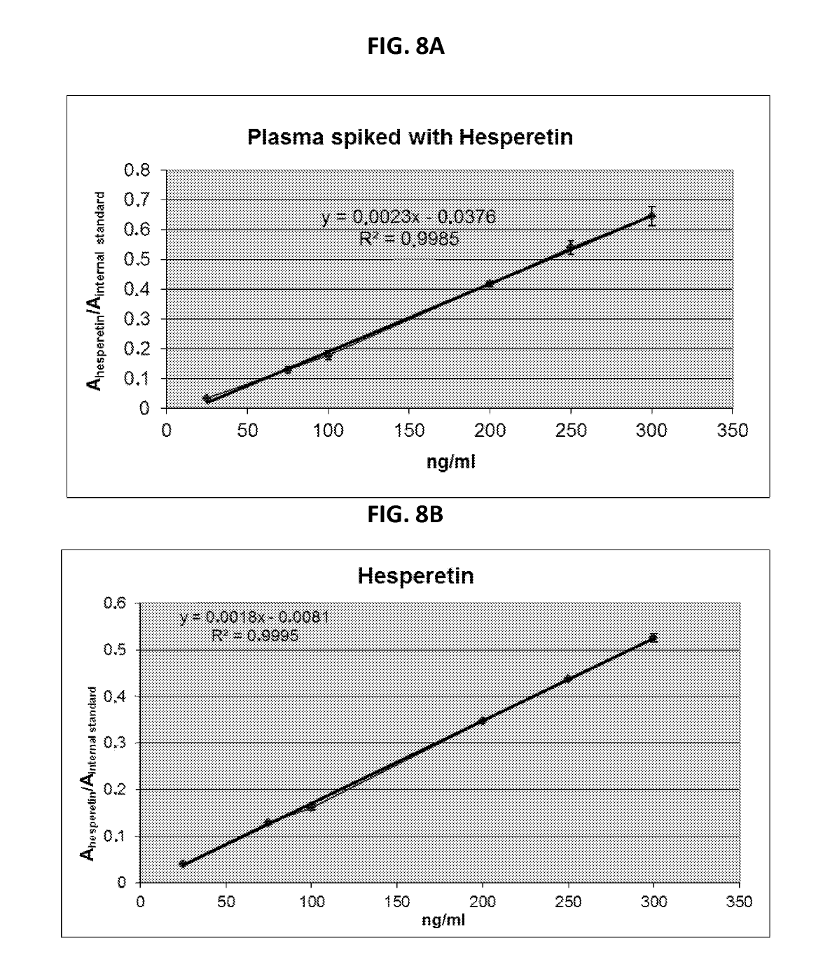

Clean chromatograms were obtained without interfering peaks at the retention times of hesperetin and the internal standard (FIGS. 7A and B). The linearity of the method was demonstrated over the concentration range of 25-300 ng/ml of hesperetin, by assaying six working solutions (n=4) and six calibration standard samples (n=4). Calibration curves were obtained by plotting the peak height ratios of hesperetin/internal standard (y) versus the hesperetin concentrations (ng/ml) in spiked plasma samples (x). By replicate analysis of calibration standard samples for the lower concentration of hesperetin a relative standard deviation (RSD) of 6.9% was obtained and for the higher concentration an RSD of 5.09% was obtained (FIG. 8A). By replicate analysis of working solutions standard for the lower concentration an RSD of 2.9% was obtained and for the higher concentration an RSD of 1.77% was obtained (FIG. 8B).

The absolute recovery of hesperetin was assessed by direct comparison of peak heights from calibration standard samples versus those found by direct injection of standards of the same concentration prepared in methanol/water (1:1). The mean recovery for hesperetin was 83.56±5.77, 109.84±9.34 and 123.47±5.18 at the 25, 100 and 250 ng/ml concentrations, respectively.

### Example 4

### Pharmacokinetics of Hesperidin or Hesperetin

This example describes representative methods for determining a pharmacokinetic profile of oral hesperidin or hesperetin in a subject.

Subjects (for example normal or obese subjects) are administered a single oral dose of 500 mg hesperidin or hesperetin in capsule form. No food is allowed until four hours after administration, when a standardized flavanone-free meal and water are provided to the subject. Blood samples (5 ml) are collected from each subject immediately before and 2, 4, 6, and 8 hours after administration.

Blood samples are centrifuged to separate plasma (for example, 3500×g for 20 minutes at 4° C.). Plasma is stored at −80° C. in appropriate buffer (such as 20% ascorbic acid and 0.4 M NaH₂PO₄ containing 0.1% EDTA, pH 3.6) until analysis. Samples are analyzed for hesperetin concentration, as described in Example 3, above. One of skill in the art can calculate pharmacokinetic parameters (such as Cₘₐₓ, Tₘₐₓ, AUC₍₀₋ₜ₎, t_{1/2}, and Rₘₐₓ) and utilize these parameters to select appropriate doses of hesperidin or hesperetin for use in treating a subject, for example a subject with metabolic syndrome and/or insulin resistance.

### Example 5

### Clinical Trial Treating Metabolic Syndrome or Insulin Resistance with Purified Hesperetin

This example describes exemplary methods for treating a subject with metabolic syndrome or insulin resistance using purified hesperetin. One skilled in the art will appreciate, based on the teachings provided herein, that methods that deviate from these specific methods can also be used to successfully treat metabolic syndrome or insulin resistance.

In one example, a clinical trial includes half (or some other proportion) of the subjects following an established protocol for treatment of metabolic syndrome or insulin resistance, or alternatively, a placebo control. The other half (or other proportion) is treated by administering purified hesperetin.

A therapeutically effective dose of purified hesperetin is administered to the subject (such as a subject either at risk for developing metabolic syndrome or known to have metabolic syndrome). Additional agents (such as lipid lowering agents or antihypertensive agents) can also be administered to the subject simultaneously, prior to, or following administration of the purified hesperetin. Administration of purified hesperetin can be achieved by any method known in the art, such as oral, inhalation, intravenous, intramuscular, intraperitoneal, or subcutaneous administration. In a particular example, purified hesperetin is administered orally.

The amount of purified hesperetin administered to treat metabolic syndrome and/or insulin resistance depends on the subject being treated, the severity of the disorder, and the manner of administration of the therapeutic composition. Ideally, a therapeutically effective amount of purified hesperetin is the amount sufficient to prevent, reduce, and/or inhibit, and/or treat metabolic syndrome (e.g., decrease triglycerides, increase HDL, decrease blood pressure, or decrease insulin resistance) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a daily, weekly, or monthly repeated administration protocol). In one example, a therapeutic agent that includes a purified hesperetin is administered orally to a subject. As such, these compositions may be formulated with an inert diluent or with a pharmaceutically acceptable carrier. Administration of the therapeutic compositions can be taken long term (for example over a period of weeks, months or years).

Following the administration of one or more therapies, subjects having metabolic syndrome can be monitored for reductions in one or more clinical symptoms associated with metabolic syndrome or subjects having insulin resistance can be monitored for one or more indicator of insulin resistance. In particular examples, subjects are analyzed one or more times, for example prior to (baseline) and following treatment. Subjects can also be monitored one or more times during the course of treatment (for example at least 1 week, 2 weeks, 3 weeks, 4 weeks, or more following the start of administration of the purified hesperetin). Subjects can be monitored using any method known in the art. For example, blood pressure and plasma lipids (such as total cholesterol, triglycerides, HDL, and LDL) can be measured using routine tests. Insulin resistance can be measured using standard methods (such as oral glucose tolerance test, glucose clamp methods, modified insulin suppression test, homeostatic model assessment, or quantitative insulin sensitivity check index (QUICKI)). Circulating endothelial adhesion markers (such as vascular cell adhesion molecule, intracellular adhesion molecule, and soluble E-selectin) can be measured by immunoassay (such as ELISA). Proinflammatory markers (for example, C reactive protein, serum amyloid A protein, and homocysteine) and/or prothrombotic markers (such as PAI-1 or fibrinogen) can also be measured by standard methods, such as immunoassay. A reduction in the clinical symptoms associated with metabolic syndrome, for example, decreased blood pressure, decreased triglycerides, increased HDL, or decreased insulin resistance (e.g., decreased fasting plasma glucose or decreased fasting plasma insulin) indicates the effectiveness of the treatment. A reduction in circulating endothelial adhesion markers and/or pro-inflammatory markers also indicates the effectiveness of the treatment. A reduction in one or more indicator of insulin resistance (such as decreased blood glucose levels, decreased blood insulin levels) indicates effectiveness of the treatment.

One of skill in the art will appreciate that the disclosed agents including purified hesperetin can be tested for safety in animals, and then used for clinical trials in animals or humans. In one example, animal models of metabolic syndrome are employed to determine therapeutic value of the disclosed agents.

### Example 6

### Methods of Treating Metabolic Syndrome or Insulin Resistance with Purified Hesperetin

This example describes methods that can be used to treat metabolic syndrome and/or insulin resistance in a subject. One skilled in the art will appreciate, based on the teachings provided herein, that methods that deviate from these specific methods can also be used to successfully treat metabolic syndrome or insulin resistance.

In an example, a subject who has been diagnosed with metabolic syndrome and/or insulin resistance is identified. Following subject selection, a therapeutically effective dose of a composition including purified hesperetin is administered to the subject. In an example, the composition includes hesperetin with a purity of at least 75% by weight (such as at least 80%, 85%, 90%, 95%, or more purity by weight). The amount of the composition administered to prevent, reduce, inhibit, and/or treat metabolic syndrome and/or insulin resistance depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., metabolic syndrome and/or insulin resistance) in a subject without causing a substantial cytotoxic effect in the subject.

In one specific example, purified hesperetin is administered orally. For example, purified hesperetin is administered at about 1-25 mg/kg daily for at least 3 weeks. In another example, purified hesperetin is administered for at least 3 weeks at a dosage that results in a hesperetin peak plasma concentration (Cₘₐₓ) of about 0.5-5 μM in the subject.

A reduction in the clinical symptoms associated with metabolic syndrome, for example, decreased blood pressure, decreased triglycerides, increased HDL, or decreased insulin resistance (e.g., decreased fasting plasma glucose or decreased fasting plasma insulin or decreased QUICKI score) indicates the effectiveness of the treatment. A reduction in circulating endothelial adhesion markers and/or pro-inflammatory markers also indicates the effectiveness of the treatment. A reduction in one or more indicator of insulin resistance (such as decreased blood glucose levels and/or decreased blood insulin levels) indicates effectiveness of the treatment.

### Example 7

### Clinical Trial Treating Metabolic Syndrome or Insulin Resistance with Purified Hesperidin

This example describes exemplary methods for treating a subject with metabolic syndrome or insulin resistance with purified hesperidin. One skilled in the art will appreciate based on the teachings herein that methods that deviate from these specific methods can also be used to successfully treat metabolic syndrome or insulin resistance.

The clinical trial is a randomized double-blind, placebo-controlled trial of oral hesperidin therapy (500 mg once daily for 3 weeks) in healthy non-obese subjects (BMI 22-25 kg/m²) and subjects with obesity (BMI 30-45 kg/m²). The primary prospective outcome is insulin sensitivity as assessed using the reference hyperinsulinemic euglycemic glucose clamp method. Whole body insulin sensitivity and hepatic glucose production is assessed using the reference hyperinsulinemic euglycemic glucose clamp method with tracer-labeled glucose before and after placebo or hesperidin therapy in non-obese and obese subjects. Changes in endothelial function of finger capillaries using the Endo-PAT finger plethysmography device are measured before each glucose clamp study and during the steady-state hyperinsulinemic period of the clamp. Changes in circulating markers of inflammation and insulin sensitivity as well as lipids (hsCRP, SAA, soluble E-selectin, TNF-α, leptin, adiponectin, total cholesterol, LDL cholesterol, HDL cholesterol) are assessed before and after hesperidin or placebo therapy. Pharmacokinetics of hesperidin and its metabolites are measured in plasma after hesperidin or placebo dosing before each glucose clamp study.

A reduction in insulin resistance (e.g., decreased fasting plasma glucose or decreased fasting plasma insulin) indicates the effectiveness of the treatment. An increase in endothelial function of finger capillaries indicates effectiveness of the treatment. A reduction in markers of inflammation and insulin sensitivity as well as lipids also indicates the effectiveness of the treatment.

### Example 8

### Methods of Treating Metabolic Syndrome or Insulin Resistance with Purified Hesperidin

This example describes methods that can be used to treat metabolic syndrome and/or insulin resistance in a subject. One skilled in the art will appreciate based on the teachings herein that methods that deviate from these specific methods can also be used to successfully treat metabolic syndrome or insulin resistance.

A subject who has been diagnosed with metabolic syndrome and/or insulin resistance is identified and selected. Alternatively, a subject with obesity (BMI>30 kg/m²) and one or more of high blood pressure, increased atherogenic markers, increased inflammatory markers, or history of at least one heart attack is identified and selected. Following subject selection, a composition including 1-3 mg/kg purified hesperidin is administered to the subject orally for at least 3 weeks.

A reduction in the clinical symptoms associated with metabolic syndrome, for example, decreased blood pressure, decreased triglycerides, increased HDL, or decreased insulin resistance (e.g., decreased fasting plasma glucose or decreased fasting plasma insulin or decreased QUICKI score) indicates the effectiveness of the treatment. A reduction in circulating endothelial adhesion markers and/or pro-inflammatory markers also indicates the effectiveness of the treatment. A reduction in one or more indicator of insulin resistance (such as decreased blood glucose levels and/or decreased blood insulin levels) indicates effectiveness of the treatment.

This disclosure provides methods of treating a subject with metabolic syndrome by administering a therapeutically effective amount of a citrus flavanone, such as purified hesperetin or purified hesperidin. The disclosure further provides methods of treating a subject with insulin resistance by administering a therapeutically effective amount of a citrus flavanone, such as purified hesperetin or purified hesperidin. It will be apparent that the precise details of the methods described may be varied or modified without departing from the spirit of the disclosure. We claim all such modifications and variations that fall within the scope and spirit of the claims below.

We claim:

1. A method of treating metabolic syndrome in a subject, comprising administering a therapeutically effective amount of a composition comprising purified hesperetin to the subject, thereby treating metabolic syndrome in the subject.

2. A method of treating metabolic syndrome in a subject, comprising administering a therapeutically effective amount of a composition comprising 1.0 mg/kg to 3.0 mg/kg purified hesperidin to the subject, thereby treating metabolic syndrome in the subject.

3. The method of claim 1 or claim 2, wherein treating metabolic syndrome comprises decreasing triglyceride levels, increasing high density lipoprotein levels, decreasing blood pressure, decreasing fasting blood glucose, or a combination of two or more thereof in the subject as compared with a control.

4. A method of treating insulin resistance in a subject, comprising administering a therapeutically effective amount of a composition comprising purified hesperetin to the subject, thereby treating insulin resistance in the subject.

5. A method of treating insulin resistance in a subject, comprising administering a therapeutically effective amount of a composition comprising 1.0 mg/kg to 3.0 mg/kg purified hesperidin to the subject, thereby treating insulin resistance in the subject.

6. The method of claim 4 or claim 5, wherein treating insulin resistance comprises decreasing plasma glucose levels, decreasing plasma insulin levels, or a combination thereof in the subject as compared with a control.

7. The method of any one of claims 4 to 6, wherein insulin resistance is determined by reference glucose clamp, homeostatic model assessment, or quantitative insulin sensitivity check index.

8. The method of any one of claims 1 to 7, wherein the purified hesperetin has a purity of greater than about 75%.

9. The method of claim 8, wherein the purity is greater than about 90%.

10. The method of any one of claims 1 to 9, wherein the composition is administered orally.

11. The method of claim 10, wherein the composition comprises a nanodispersion.

12. The method of claim 11, wherein the nanodispersion comprises polyvinylpyrrolidone.

13. The method of any one of claims 1 to 12, wherein the composition further comprises a pharmaceutically acceptable carrier.

14. The method of claim 13, wherein the composition consists essentially of purified hesperetin and a pharmaceutically acceptable carrier.

15. The method of claim 13, wherein the composition consists essentially of purified hesperidin and a pharmaceutically acceptable carrier.

16. The method of claim 1 or claim 4, wherein the purified hesperetin comprises hesperetin aglycone or hesperetin-7-glucoside.

17. The method of claim 1 or claim 4, wherein the therapeutically effective amount comprises an amount of purified hesperetin sufficient to achieve a plasma concentration of about 0.5-10 μM in the subject.

18. The method of claim 17, wherein the amount of purified hesperetin is about 0.5-20 mg/kg purified hesperetin.

19. The method of claim 18, wherein the amount of purified hesperetin is about 1-3 mg/kg.

20. The method of claim 2 or claim 5, wherein the therapeutically effective amount comprises an amount of purified hesperidin sufficient to achieve a plasma concentration of hesperetin of about 0.5-10 μM in the subject.

21. The method of any one of claims 1 to 20, further comprising selecting a subject with obesity and one or more of increased blood pressure, increased circulating atherogenic markers, increased circulating inflammatory markers, increased circulating cell adhesion markers, or history of heart attack.

22. The method of claim 21, wherein the circulating inflammatory markers comprise one or more of C-reactive protein, serum amyloid A protein, and homocysteine.

23. The method of claim 21, wherein the circulating atherogenic markers comprise one or more of plasminogen activator inhibitor-1 protein, monocyte chemoattractant protein 1, fibrinogen, and/or serum amyloid A protein.

24. The method of claim 21, wherein the circulating cell adhesion markers comprise one or more of vascular cell adhesion molecule 1, intracellular adhesion molecule 1, or E-selectin.
